(19) United States
(12) Patent Application Publication
(10) Pub. No.: US 2009/0029372 A1
(43) Pub. Date: Jan. 29, 2009

(54) ADAM12 AS A BIOMARKER FOR BLADDER CANCER

(75) Inventor: Ulla M. Wewer, Klampenborg (DK)

Correspondence Address: KNOBBE MARTENS OLSON & BEAR LLP, 2040 MAIN STREET, FOURTEENTH FLOOR, IRVINE, CA, 92614, US

(73) Assignee: Kobenhavns Universitet, Copenhagen (DK)

(21) Appl. No.: 12/120,544

(22) Filed: May 14, 2008

Related U.S. Application Data
(60) Provisional application No. 60/917,705, filed on May 14, 2007.

(51) Int. Cl. C12Q 1/68; G01N 33/573

(52) U.S. Cl. 435/6; 435/7.4

(57) ABSTRACT

The present inventors have shown that the gene and protein expression profiles of ADAM8, ADAM10 and ADAM12 in different grades and stages of bladder cancer.

ADAM12 gene expression was evaluated in tumors from 96 patients with bladder cancer using a customized Affymetrix GeneChip. Gene expression in bladder cancer was validated using reverse transcription-polymerase chain reaction (RT-PCR), quantitative PCR, and in situ hybridization. Protein expression was evaluated by immunohistochemical staining on tissue arrays of bladder cancers.

The presence and relative amount of ADAM12 in the urine of cancer patients were determined by Western blotting and densitometric measurements, respectively.

Particularly ADAM12 mRNA expression was significantly upregulated in bladder cancer, as determined by microarray analysis, and the level of ADAM12 mRNA correlated with disease stage. ADAM12 protein expression correlated with tumor stage and grade. ADAM12 was present in higher levels in the urine from bladder cancer patients than in urine from healthy individuals. Significantly, following removal of tumor by surgery, in most bladder cancer cases examined the level of ADAM12 in the urine decreased and, upon recurrence of tumor, increased.

### CROSS-REFERENCE TO RELATED APPLICATIONS

This application claims the benefit of priority to U.S. Provisional Patent Application No. 60/917,705; filed May 14, 2007, the disclosure of which is hereby expressly incorporated by reference in its entirety.

### FIELD OF THE INVENTION

Aspects of the invention concern the field of cancer diagnostics. More specifically, it has been discovered that certain genes are upregulated in bladder cancer. Provided herein are methods of diagnosis or prognosis of bladder cancer or methods of determining the potential to acquire bladder cancer by detecting the presence or absence of a biological marker, desirably ADAM8 or ADAM10, and preferably, ADAM12, in a biological sample, such as urine.

### BACKGROUND OF THE INVENTION

Bladder cancer is a common malignant disease in both men and women; with estimates projecting that over 60,000 new cases would be diagnosed yearly in United States alone. The majority of patients diagnosed with bladder cancer present with non-muscle invasive tumors. However, despite intensive surveillance, these patients have a risk of progression of the disease to muscle invasive cancer of up to 60% at long-term follow-up.

ADAM12 is a protease, and proteases have multiple functions in normal and pathophysiological conditions. Matrix metalloproteases (MMPs) have been studied extensively, and increased activity of these proteolytic enzymes has been shown to be associated with the malignant phenotype. More recently, the ADAM family of proteins, including ADAM9, 12 and 28, has been implicated in cancer.

The present inventors have earlier reported that ADAM12 is highly expressed by the malignant tumor cells in several different forms of cancer. The present inventors e.g. reported that ADAM12 mRNA was almost undetectable in normal livers, but increased in hepatocellular carcinomas (a six-fold increase) and liver metastases from colonic carcinomas (up to a 60-fold increase).

ADAM12 is selectively overexpressed in glioblastomas, with a direct correlation between the level of ADAM12 mRNA expression and cell proliferation activity. In situ hybridization and immunohistochemical analysis demonstrated that ADAM12 is produced by the glioblastoma cells.

The present inventors and others have studied ADAM12 in breast cancer and found that urinary levels of ADAM12 correlate with breast cancer status and stage (Roy R et al.).

Most recently, the present inventors demonstrated that ADAM12 enhances mammary tumor progression in a transgenic mouse model. When ADAM12 expression was increased, time of tumor onset was decreased and tumor burden, metastasis, and grade of malignancy were increased. The present inventors also provided evidence that ADAM12 decreases apoptosis of tumor cells and enhances apoptosis of stromal cells.

WO 06/121710 discloses several other differentially expressed genes in bladder cancer.

WO 06/91412 demonstrates that ADAMTS-7 is a marker for cancers of general epithelial origin.

However, the current procedure for detecting bladder tumors with potential progression is difficult and error-prone, and new biomarkers are needed to optimize the molecular characterization of tumors.

### SUMMARY OF THE INVENTION

In this application, the present inventors investigated the potential of e.g. ADAM12 as a biomarker in bladder cancer by demonstrating that both the mRNA and protein expression levels of ADAM12 correlate with the stage of bladder cancer.

The present inventors also demonstrated that ADAM12 levels in the urine from bladder cancer patients are significantly increased as compared to urine from healthy individuals.

Importantly, the present inventors found that the level of ADAM12 in urine decreased following tumor removal and increased upon tumor recurrence, suggesting that ADAM12 could become an important biomarker for bladder cancer diagnostics and surveillance.

One aspect of the invention relates to a method for screening an individual for bladder cancer said method comprising the steps of:
a) obtaining a sample from an individual
b) determining the level of ADAM12 in said sample by detecting
c) comparing said level with a reference level;
d) identifying whether the level is different from said reference level and
   evaluating whether the individual has an increased risk of bladder cancer, if the level is higher than the reference level.

The inventors further discloses that ADAM8 and ADAM10 can work as a biomarker for bladder cancer in a similar manner as ADAM12, thus it should be understood that any feature and/or aspect discussed above in connection with the methods describing ADAM12 apply by analogy to methods describing ADAM8 and/or ADAM10 according to the present invention.

In another aspect the invention relates to a method for determining whether an individual is likely to have recurrent bladder cancer after being treated for bladder cancer.

Accordingly, aspects of the invention concern a method for screening an individual for bladder cancer said method comprising the steps of:
a) obtaining a sample from the individual,
b) determining the level of ADAM12 in said sample,
c) comparing said level with a reference level,
d) identifying whether the level is different from said reference level and
   evaluating the disease status of the individual or whether the individual has an increased risk of bladder cancer, if the level is higher than the reference level.

More aspects of the invention concern a method for screening an individual for bladder cancer said method comprising the steps of:
a) determining the level of ADAM12 in a sample obtained from the individual,
b) constructing a percentile plot of said level of ADAM12 obtained from a healthy population,
c) constructing a ROC (receiver operating characteristics) curve based on the ADAM12 level determined in the healthy population and on the ADAM12 level determined in a population who has developed bladder cancer,
d) selecting a desired specificity,
e) determining from the ROC curve the sensitivity corresponding to the desired specificity,
f) determining from the percentile plot the ADAM12 level corresponding to the determined sensitivity; and
g) predicting the individual to have bladder cancer, if the ADAM12 level in the sample is equal to or higher than said ADAM12 level corresponding to the determined specificity and predicting the individual as unlikely or not to having bladder cancer if the ADAM12 level in the sample is lower than said ADAM12 level corresponding to the determined specificity.

More aspects of the invention concern a method for screening an individual for bladder cancer said method comprising the steps of:
a) determining the level of ADAM12 in a sample obtained from the individual,
b) constructing a percentile plot of said level of ADAM12 obtained from a healthy population,
c) selecting a desired sensitivity,
d) determining from the percentile plot the ADAM12 level corresponding to the desired sensitivity; and
e) predicting the individual to have bladder cancer, if the ADAM12 level in the sample is equal to or higher than said ADAM12 level corresponding to the determined specificity and predicting the individual as unlikely or not to having bladder cancer if the ADAM12 level in the sample is lower than said ADAM12 level corresponding to the determined specificity.

More aspects of the invention concern a method for determining whether an individual is likely to have recurrent bladder cancer after being treated for bladder cancer, said method comprises the steps of:
a) determining the level of ADAM12 in a sample obtained post-treatment from the individual treated for bladder cancer
b) comparing said level with a reference level;
c) identifying whether the level is different from said reference level and
   evaluating whether the individual is likely to have recurrent bladder cancer, if the level is higher than the reference level.

More aspects of the invention concern the above methods, wherein the level of ADAM12 is a combined level of ADAM12 selected from the group consisting of any combination of the level of ADAM12 polypeptide, the level of polynucleotide encoding ADAM12 and the level of specific ADAM12 protease activity.

More aspects of the invention concern the above methods, wherein said method can differentiate between different grades and stages of bladder cancer.

More aspects of the invention concern the above methods, wherein the ADAM12 level is determined by determining ADAM12-L.

More aspects of the invention concern the above methods, wherein the ADAM12 level is determined by determining ADAM12-S.

More aspects of the invention concern the above methods, wherein the ADAM12 level is determined by measuring ADAM12 mRNA or DNA.

More aspects of the invention concern the above methods, wherein the ADAM12 level is determined on protein level.

More aspects of the invention concern the above methods, wherein the sample is urine, blood or plasma.

More aspects of the invention concern the above methods, wherein the sample is a tissue biopsy.

More aspects of the invention concern the above methods, wherein said determination of the level is carried out on a DNA array.

More aspects of the invention concern the above methods, wherein the ADAM12 level is combined with values from at least one other combinatorial marker.

More aspects of the invention concern the above method, wherein the combinatorial marker is selected from the group consisting of ADAM8, ADAM10, MMP2 and MMP9.

More aspects of the invention concern an array comprising a nucleic acid which binds ADAM12 for the determination of bladder cancer.

More aspects of the invention concern a method for treating bladder cancer comprising:
identifying a mammal expressing elevated levels of ADAM12, and
administering to said mammal an effective amount of a drug sufficient to reduce tumor growth or prevent metastasis.

More aspects of the invention concern a method for diagnosing an individual for bladder cancer said method comprising the steps of:
a) obtaining a sample from said individual
b) determining the level of ADAM12 in said sample
c) comparing said level with a reference level;
d) identifying whether the level is different from said reference level and
   evaluating whether the individual has bladder cancer, if the level is higher than the reference level.

More aspects of the invention concern a method for screening an individual for bladder cancer said method comprising the steps of:
a) providing a sample from the individual
b) determining the nucleotide level of ADAM8 in said sample
c) comparing said level with a reference level;
d) identifying whether the level is different from said reference level and
   evaluating the disease status of the individual or whether the individual has an increased risk of bladder cancer, if the level is higher than the reference level.

More aspects of the invention concern a method for screening an individual for bladder cancer said method comprising the steps of:
a) providing a sample from an individual
b) determining the nucleotide level of ADAM10 in said sample
c) comparing said level with a reference level;
d) identifying whether the level is different from said reference level and
   evaluating the disease status of the individual or whether the individual has an increased risk of bladder cancer, if the level is higher than the reference level.

More aspects of the invention concern a method for screening an individual for the presence of an epithelial cancer said method comprising the steps of:
a) obtaining a sample from the individual,
b) determining the level of ADAM12 in said sample,
c) comparing said level with a reference level,
d) identifying whether the level is different from said reference level and
   evaluating the disease status of the individual or whether the individual has an increased risk of an epithelial cancer, if the level is higher than the reference level.

### BRIEF DESCRIPTION OF THE DRAWINGS

FIG. 1 Gene expression profiling of ADAM12 in bladder cancer.

(A) Microarray analysis of ADAM12-L gene expression levels in 21 normal bladder mucosa samples, 31 Ta tumors, 20 T1 tumors, and 45 T2-4 tumors. (B) RT-PCR analysis of mRNA expression of human ADAM12-L, ADAM12-S, and GADPH in normal bladder mucosa tissue (lanes 1-3) and Ta (lanes 4-8) and T2-4 (lanes 9-13) bladder cancer. (C) Quantitative PCR analysis of ADAM12-L mRNA expression in two normal bladder mucosa samples, six Ta, and five T2-4 tumors. In A and C, the horizontal lines represent the average expression intensity in each group.

FIG. 2 In situ hybridization of ADAM12 in bladder cancer.

(A) Tumor sections from ADAM12-MMTV-PyMT mouse breast cancer tissue. Strong hybridization signal with ADAM12 T3 anti-sense probe is present as dark grains over the tumor islets and only very weak signals are seen over the surrounding stroma. (B) Dark field image of the same tumor area as in panel A. (C) Tumor sections from human bladder cancer (grade 2) with positive signal over the tumor cell (T) using T3 anti-sense probe. (D) Dark field image of the same tumor as in panel C. (E) Adjacent tumor sections from the same bladder tumor as in C, D with ADAM12 T7 sense probe hybridization with little or no signal. (F) Dark field image of the same tumor area as in panel E. Bar in panel B=20 μm and in panel F=8 μm. Panels A and B are the same magnification and panels C-F are the same magnification.

FIG. 3 ADAM12 immunostaining of bladder cancer tissue arrays.

Tissue sections were incubated with a polyclonal antibody to human ADAM12 (rb122), then detection performed with a streptavidin-biotin technique. (A) Non-muscle invasive papillary bladder cancer (T1, grade 1) with strong positive staining for ADAM12 in most of the tumor cells. (B) Non-muscle invasive papillary bladder cancer (T1, grade 2) with uniform ADAM12 cytoplasmic immunostaining confined to the perinuclear Golgi-like area. (C) Invasive bladder cancer (T2, grade 2) with ADAM12 immunostaining localized mostly along the plasma membranes. (D) Invasive bladder cancer (T3, grade 3) with ADAM12 immunostaining in the cytoplasm in some cells while other tumor cells are less intensively stained. (E) Invasive bladder cancer (T3, grade 3) with strong ADAM12 staining of tumor cells along the invasive front of the tumor. (F) Invasive bladder cancer (T3, grade 3) with strong ADAM12 immunostaining of tumor cells located inside the blood vessels. (G) ADAM12 immunostaining and correlation to tumor grade 1-3 (histopathological diagnosis). The number of grade 3 tumor cases (%) positive for ADAM12 staining is significantly higher than the number of grade 1 tumor cases positive for ADAM12, p<0.005 (Chi-square; Pearson). (H) ADAM12 immunostaining and correlation to tumor stage (TNM). The number of T2-T4 tumor cases (%) positive for ADAM12 staining is significantly higher than the number of Ta-T1 tumor cases positive for ADAM12, p<0.00001 (Chi-square; Pearson). Sections were counterstained with hematoxylin. Bar in panel A=20 μm and F=10 μm. Panels A, C are the same magnification and panels B, D-F are the same magnification.

FIG. 4 ADAM12 immunostaining of normal and dysplastic bladder mucosa.

Tissue sections were incubated with polyclonal antibodies to human ADAM12, then detection performed with a streptavidin-biotin technique. (A) Normal bladder urothelium exhibited weak ADAM12-S staining with rb116 and (B) no ADAM12 immunoreactivity with preimmune serum. (C) The umbrella cells exhibited strong ADAM12 positive staining. (D) The apical surface of umbrella cells also stained with antibodies to uroplakin 3, an umbrella cell marker. (E) Squamous epithelial cells isolated from the urine did not exhibit ADAM12 immunostaining, whereas (F) the umbrella cells in the urine exhibited strong ADAM12 immunostaining (rb122). Note the larger nuclei of the umbrella cells compared to the squamous cells. (G) Atypical hyperplasia showed strong ADAM12 immunostaining in the umbrella cells, whereas the underlying epithelium exhibited only weak staining. (H) Larger magnification of the one of the umbrella cells shown in G. Note the strong immunoreaction, particularly along the cell periphery. (I) Carcinoma in situ exhibited intense ADAM12 immunostaining of the epithelial cells. (3) Transitional cell carcinoma (grade 2) demonstrated the strongest ADAM12 immunostaining in the most non-muscle invasive tumor cells that mimic the morphology of umbrella cells (named “umbrella-cell differentiation”). This staining pattern was found in 23 out of 155 cases of bladder tumors (14.8%) examined in this study. Sections were counterstained with hematoxylin. Bar in panel D=7 μm, F=5 μm, H=7 μm, I=8 μm, J=20 μm. Panels A, B, E, F are the same magnification, panels C, D are the in same magnifications, and panels G and 3 are the same magnifications.

FIG. 5 Western blotting analysis of ADAM12 in urine from normal controls and bladder cancer patients.

(A) Urine from normal controls (lane 1) and from two patients with a T2-4 tumor (lane 2, 3) prepared using reducing or nonreducing conditions were loaded onto SDS-PAGE gels, transferred to Immobilon-P, and probed with a mixture of polyclonal antibodies against human ADAM12 (one directed against the cysteine-rich domain (rb122) and the other the prodomain (rb132)), or a monoclonal antibody against ADAM12 (6E6). The 68 and 27 kDa bands represent the mature form and the prodomain of ADAM12, respectively. (B) Immunoprecipitate of urine supernatant using a mixture of monoclonal antibodies (6E6, 8F8, 6C10) against ADAM12 (lane1) and purified ADAM12-S (lane 2) were immunoblotted with a mixture of antibodies against the carboxy-terminus and the prodomain of ADAM12-S (rb116, rb132). (C) Estimate of the relative amount of ADAM12 in urine supernatant using purified ADAM12-S as standard. 40 μg protein was loaded per lane (for the pool of normal urine (np) 6 μl was loaded, and for the two T2-4 patients (pt 1 and pt 2) 12 μl and 4 μl was loaded, respectively). Urine samples were immunoblotted using a mixture of polyclonal antibodies rb122 and rb132. (D) Representative urine samples (40 μg protein per lane) from normal controls (upper panel), patients with Ta tumors (middle panel), and patients with T2-4 tumors (lower panel) were immunoblotted with rb122. The protein band represents the mature form of ADAM12-S at 68 kDa. On all Western blots, a pool of normal urine is presented in the first lane (np). (E) Densitometric quantitation of the ADAM12 68 kDa band signal present in urine from eight normal volunteers, 11 patients with Ta tumors, four patients with T1 tumors, and 17 patients with T2-4 tumors. The pool of normal urine (np) was used to normalize the apparent amount of ADAM12 in normal and cancer urine. The data represent triplicate experiments with error bars denoting standard errors. *p=0.0004, **p=0.0001, ***p=0.00021 (Student's t-test).

FIG. 6 Western blotting analysis of ADAM12 in urine of bladder cancer patients who underwent surgical removal of tumor.

Urine samples were subjected to immunoblotting using rb122 and densitometric quantitation of the resulting 68 kDa ADAM12 band was performed. (A) In the upper panel, urine from a pool of normal controls (np, lane 1), and from a patient (case A) with non-muscle invasive bladder cancer prior to transurethral resection (Ta tumor, lane 2), during the surveillance period in which no tumor could be detected (tumor free, lane 3), and when recurrence of invasive tumor was diagnosed (T2-4, lane 4). Forty μg of total protein was applied per lane. In the lower panel, the pool of normal urine was used to normalize the apparent amount of ADAM12 in the cancer urine. (B) The relative amount of ADAM12 in the urine from six cases of bladder cancer during a follow-up study (as described in A) was quantitated (also as described in A). Averages presented are means±standard deviation.

### DETAILED DESCRIPTION OF THE INVENTION

There is a tremendous need to identify comprehensive biomarkers to predict, diagnose, and monitor disease, including cancer. ADAM proteins are members of the metzincin superfamily of zinc-dependent proteases and have been implicated in normal and abnormal growth during development and in disease, such as cancer.

In the present application, the present inventors report that the levels of ADAM8, 10, and 12 mRNAs are significantly upregulated in human bladder cancer, and the present inventors examined in more detail that particular ADAM12 is a valuable biomarker for bladder cancer. Thus the invention discloses a method for detecting, screening, monitoring and diagnosing a bladder cancer in a mammal comprising the steps of assaying a sample for an elevated level of ADAM12 and correlating an elevated level of ADAM8, ADAM10 and/or ADAM12 in said sample with bladder cancer.

It should be understood that any feature and/or aspect discussed above in connection with the “methods for screening” apply by analogy to methods of diagnosing, monitoring etc.

Thus, one aspect the present invention relates to a method for screening an individual for bladder cancer said method comprising the steps of:
a) providing a sample from an individual,
b) determining the level of ADAM12 in said sample,
c) comparing said level with a reference level
d) identifying whether the level is different from said reference level, and
   evaluating whether the individual has an increased risk of bladder cancer, if the level is higher than the reference level.

In another aspect, the invention relates to method for screening an individual for bladder cancer said method comprising the steps of:
a) obtaining a sample from the individual,
b) determining the level of ADAM12 in said sample,
c) comparing said level with a reference level,
d) identifying whether the level is different from said reference level and
   evaluating the disease status of the individual or whether the individual has an increased risk of bladder cancer, if the level is higher than the reference level As mentioned above ADAM8 and ADAM10 can also work as a biomarker for bladder cancer in a similar manner as ADAM12, thus it should be understood that any feature and/or aspect discussed above in connection with the methods describing ADAM12 apply by analogy to methods describing ADAM8 and/or ADAM10 according to the present invention.

The “methods” of the present invention may include, but is not limited to determining the metastatic potential of a tumor or determining a patient's prognosis following discovery of a tumor. Such methods may also be used for determining the effectiveness of a therapeutic regime used to treat cancer or other disease involving the presence of elevated levels of any of the markers described herein.

As mentioned, the terms “diagnostic method” or “monitoring method” or “screening method” or “prognostic method” are used interchangeably.

In another aspect the present invention discloses a method for screening an individual for bladder cancer said method comprising the steps of:
a) providing a sample from the individual
b) determining the nucleotide level of ADAM8 in said sample
c) comparing said level with a reference level;
d) identifying whether the level is different from said reference level and
   evaluating the disease status of the individual or whether the individual has an increased risk of bladder cancer, if the level is higher than the reference level.

In one aspect, the present invention discloses a method for screening an individual for bladder cancer said method comprising the steps of:
a) providing a sample from an individual
b) determining the nucleotide level of ADAM10 in said sample
c) comparing said level with a reference level;
d) identifying whether the level is different from said reference level and
   evaluating the disease status of the individual or whether the individual has an increased risk of bladder cancer, if the level is higher than the reference level.

Those skilled in the art may combine the biomarkers described herein with further known cancer markers to evaluate the whether the individual has cancer or not.

Further, the skilled addressee may used any of the ADAMs disclosed herein individually or in combination to improve the methods described, thus in one embodiment the present invention described a method for screening an individual for bladder cancer said method comprising the steps of:
a) providing a sample from an individual
b) determining the level of at least on of the biomarkers selected from the group consisting of ADAM8, ADAM10 and ADAM12 in said sample
c) comparing said level with a reference level;
d) identifying whether the level is different from said reference level and
   evaluating whether the individual has an increased risk of bladder cancer, if the level is higher than the reference level.

Human ADAM12 is produced in two splice variants, the prototype transmembrane ADAM12-L and the shorter secreted ADAM12-S. The bladder cancer microarray the present inventors used only allowed us to determine the expression levels of ADAM12-L, whereas for unknown reasons ADAM12-S was not detected. However, ADAM12-S mRNA could be detected from bladder cancer tissue by RT-PCR. The present inventors therefore conclude that bladder cancers express both ADAM12-L and ADAM12-S. This is consistent with previous studies that found that levels of RNA for both forms of ADAM12 were increased in cirrhosis, hepatocelluar carcinomas, and liver metastases from colorectal cancers compared to normal controls.

### Bladder Cancer

In one embodiment, a method for facilitating the diagnosis of cancer of epithelial origin such as bladder cancer in a patient is provided. The method comprises obtaining a biological sample from an individual and detecting the presence or absence of ADAM12 or a fragment thereof in the biological sample, wherein the presence of ADAM12 or elevated levels of ADAM12 is indicative of the presence of cancer of epithelial origin. In the present context, the cancer of epithelial origin may be selected from the group consisting of breast cancer, basal cell carcinoma, adenocarcinoma, gastrointestinal cancer, lip cancer, mouth cancer, esophageal cancer, small bowel cancer, stomach cancer, colon cancer, liver cancer, brain, bladder cancer, pancreas cancer, ovary cancer, cervical cancer, lung cancer, skin cancer, prostate cancer, and renal cell carcinoma.

In another embodiment the invention discloses a method for screening an individual for the presence of an epithelial cancer said method comprising the steps of:
a) obtaining a sample from the individual,
b) determining the level of ADAM12 in said sample,
c) comparing said level with a reference level,
d) identifying whether the level is different from said reference level and
   evaluating the disease status of the individual or whether the individual has an increased risk of an epithelial cancer, if the level is higher than the reference level.

As used herein, the term “bladder cancer” refers to a disease in which the cells lining the urinary bladder lose the ability to regulate their growth resulting in a mass of cells that may form a tumor, but also terms currently used in the art such as but not limited to “early bladder cancer” or “superficial bladder cancer” referring to non invasive bladder tumours (e.g. type Ta or Tia as determined in accordance with the AJCC guidelines) (Herr et al. 2001) is comprised in the present wording.

Non-muscle invasive bladder tumors can be successfully removed by transurethral resections, but the recurrence rate is high (30% to 70%), and the progression rate of superficially invasive cancer (T1) to muscle-invasive cancer (T2-4) is up to 60% in long-term follow-up. Extensive research has been undertaken to define biomarkers in urine that could either add to or replace cytology in follow-up for low-grade/stage bladder tumors.

### The Sample

In the present context, the term “sample” relates to any liquid or solid sample collected from an individual to be analyzed. Preferably, the sample is liquefied at the time of assaying.

In another embodiment of the present invention, a minimum of handling steps of the sample is necessary before measuring the level of ADAM12. In the present context, the subject “handling steps” relates to any kind of pre-treatment of the liquid sample before or after it has been applied to the assay, kit or method. Pre-treatment procedures includes separation, filtration, dilution, distillation, concentration, inactivation of interfering compounds, centrifugation, heating, fixation, addition of reagents, or chemical treatment.

In accordance with the present invention, the sample to be analyzed is collected from any kind of mammal, including a human being, a pet animal, a zoo animal and a farm animal.

In yet another embodiment of the present invention, the sample is derived from any source such as body fluids.

Preferably, this source is selected from the group consisting of milk, semen, blood, serum, plasma, saliva, urine, sweat, ocular lens fluid, cerebral spinal fluid, cerebrospinal fluid, ascites fluid, mucous fluid, synovial fluid, peritoneal fluid, vaginal discharge, vaginal secretion, cervical discharge, cervical or vaginal swab material or pleural, amniotic fluid and other secreted fluids, substances and tissue biopsies from organs such as the brain, heart and intestine.

In one embodiment of the present invention relates to a method according to the present invention, wherein said body sample or biological sample is selected from the group consisting of blood, urine, pleural fluid, oral washings, vaginal washings, cervical washings, tissue biopsies, and follicular fluid.

In one embodiment of the present invention relates to a method according to the present invention, wherein said biological sample is selected from the group consisting of blood, tissue, serum, urine, stool, sputum, cerebrospinal fluid, nipple aspirates, and supernatant from cell lysate.

Another embodiment of the present invention relates to a method, wherein said biological sample is selected from the group consisting of urine, blood, plasma and serum.

In one embodiment of the present invention relates to a method according to the present invention, wherein said sample is urine.

In one embodiment of the present invention relates to a method according to the present invention, wherein said sample is a tissue biopsy.

The sample taken may be dried for transport and future analysis. Thus the method of the present invention includes the analysis of both liquid and dried samples.

Clinical Sample—It is understood that a “clinical sample” encompasses a variety of sample types obtained from a subject and useful in the procedure of the invention, such as for example, a diagnostic, a screening test or monitoring test of ADAM8, ADAM10 or ADAM12 levels. The definition encompasses as described solid tissue samples obtained by surgical removal, a pathology specimen, an archived sample, or a biopsy specimen, tissue cultures or cells derived there from and the progeny thereof, and sections or smears prepared from any of these sources. Non-limiting examples are samples obtained from bladder tissue, lymph nodes, and bladder tumors. The definition also encompasses blood, bone marrow, spinal fluid, and other liquid samples of biologic origin, and may refer to either the cells or cell fragments suspended therein, or to the liquid medium and its solutes.

A control sample is a source of cells or tissue for comparison purposes. A control sample may include, inter alia, cancer-free tissue or an archived pathology sample containing any of the markers at various levels for use as control.

### Determining the ADAM12 Level

The determination of the level of an identified marker, such as ADAM8, ADAM10 and/or ADAM12 in a sample can be obtained by any detecting assay known to the skilled addressee, such as but not limited to immunoassays, gene expression assays and other known assays such as but not limited to arrays.

In one embodiment, the assay or a device operating said assay may be selected from the group consisting of an assay, an immunoassay, a stick, a dry-stick, an electrical device, an electrode, a reader (spectrophotometric readers, IR-readers, isotopic readers and similar readers), histochemistry, and similar means incorporating a reference, filter paper, color reaction visible by the naked eye.

The determination may be carried out on mRNA level, protein level and/or as a measurement of the protease activity of the ADAM makers described herein.

Thus, in one embodiment the level of e.g. ADAM12 is determined by at least one level selected from the group consisting of the level of ADAM12 polypeptide, the level of polynucleotide encoding ADAM12 and the level of specific ADAM12 protease activity.

In one embodiment, the ADAM12 level is determined on mRNA level.

In one embodiment, the ADAM12 level is determined by measuring ADAM12 mRNA or DNA.

In one embodiment, the ADAM12 level is determined on protein level. Human ADAM12 exists in two forms ADAM12-L (long) and ADAM12-S (short), the latter being the secreted form of ADAM12. ADAM12-S differs from ADAM12-L at the C-terminal end in that it does not contain the transmembrane and cytoplasmatic domains. ADAM12-S binds to and has proteolytic activity against insulin-like growth factor binding protein (IGFBP)-3 and, to a lesser extent, IGFBP-5. In vitro cleavage of the 44-kDa IGFBP-3 by ADAM12 yields several fragments of 10 to 20 kDa and is independent of insulin-like growth factor (IGF) I and II. IGF I and II are proinsulin-like polypeptides that are produced in nearly all fetal and adult tissues. Lack of IGF I and II causes fetal growth retardation in mice. The cleavage of IGFBPs into smaller fragments with reduced affinity for the IGFs reverses the inhibitory effects of the IGFBPs on the mitogenic and DNA stimulatory effects of the IGFs. Seventy-five percent of the IGFs are bound to IGFBP-3 in plasma.

Thus, one embodiment of the present invention relates to determination of level of ADAM12 in a sample, wherein the ADAM12 can be both the ADAM12-L (long) and ADAM12-S (short) form.

In another embodiment the ADAM12 level is determined by determining ADAM12-L.

In another embodiment the ADAM12 level is determined by determining ADAM12-S.

It is further understood by those of ordinary skill in the art, that ADAM12 is a member of a complex family of at least 33 similar genes. It is in addition possible that multiple forms of ADAM12 with small differences in amino acid sequences, or other small differences, may be synthesized. It is further possible that one or more of the ADAM12 genes are expressed, thereby producing a unique variant or variants (previously referenced as nicked or fragmented or aberrant forms) ADAM12.

According to the present invention these variants could be measured by conventional immunological techniques for measuring e.g. ADAM12. An assay produced to measure the specific ADAM12 variant, or variants, associated with bladder cancer may result in even further enhancement of detection efficiency.

Another embodiment of the present invention relates to determination of level of ADAM12 polypeptide in a sample in the form of mRNA originating from ADAM12 expression, including all splice variants of ADAM12.

Antibodies or binding reagents that specifically detect the markers disclosed herein may also be used to determine the level of the markers.

An “antibody” (interchangeably used in plural form) is an immunoglobulin molecule capable of specific binding to a target, such as a polypeptide, through at least one antigen recognition site. As used herein, the term encompasses not only intact antibodies, but also fragments thereof, mutants thereof, fusion proteins, humanized antibodies, and any other modified configuration of the immunoglobulin molecule that comprises an antigen recognition site of the required specificity. An antibody against the markers disclosed are used in the methods of the invention.

Thus in one embodiment, the determining step comprises detecting the level of ADAM12 with an antibody that recognises e.g. ADAM12.

In one embodiment the antibody may be selected from the group consisting of monoclonal antibodies and polyclonal antibodies.

In one embodiment the antibody is labelled. Such labels may be selected from the group consisting of biotin, fluorescent molecules, radioactive molecules, chromogenic substrates, chemi-luminescence and enzymes.

Additional support is provided in the results reported by Irwin et al. (2000) showing that human placental trophoblasts secrete a disintegrin and metalloprotease that cleaves IGFBP-3, is active at neutral and alkaline pH, and sensitive to o-phenanthroline. The protease secreted by trophoblasts could be ADAM12 because mRNA for ADAM12 is particularly abundant in the placenta, and has the same apparent characteristics

Thus, another embodiment of the present invention relates to determination of level of e.g. ADAM12 polypeptide in a sample, wherein said level is calculated by measuring the specific ADAM12 protease activity, preferably by detecting cleavage of IGFBP-3, a derivative thereof, or any other suitable substrate for ADAM12.

In this study, ADAM12 mRNA expression was assessed by microarray analysis for the first time. Using microarrays, the present inventors found that bladder cancers express increased amounts of ADAM12 mRNA and that the level strongly correlates with disease status.

Thus in embodiment, the present invention relates to a method as described herein wherein said determination of the level is carried out on a DNA array.

The present inventors established a qPCR method for ADAM12 that confirmed the increase of ADAM12 mRNA in bladder cancer.

Thus in embodiment, the present invention relates to a method as described herein wherein said determination of the level is carried out by qPCR.

Furthermore, in situ hybridization showed that the bladder cancer cells are the site of ADAM12 gene expression.

Thus in embodiment, the present invention relates to a method as described herein wherein said determination of the level is carried out by in situ hybridization.

Immunohistochemistry demonstrated that the protein expression pattern of ADAM12 correlates with tumor grade and stage.

Thus in embodiment, the present invention relates to a method as described herein wherein said determination of the level is carried out Immunohistochemistry.

Detection Level of ADAM12 in the Urine

In the present application, the present inventors found that while the urine level of ADAM12 was low in all healthy individuals, the urine levels of ADAM12 significantly increased in all patients with superficial non-invasive tumors (Ta), superficial invasive (T1), and were highest in patients with invasive cancers (T2-4). The present inventors also analyzed two cases of Ta tumors and four cases of T1 tumors that eventually progressed to T2-4 tumors.

The present inventors found that in most of these bladder cancer cases the level of ADAM12 in the urine decreased following surgery, was minimal during the tumor-free period, but then increased again upon recurrence of tumor. Thus, monitoring ADAM12 in the urine of bladder cancer patients might be a useful non-invasive diagnostic test, and it is possible that urinary ADAM12 could even be a marker of primary bladder cancer. Compared to cytology, measurement of ADAM12 levels was a more sensitive marker for detecting early-stage and/or low grade tumors. Cytology is known to be less sensitive in early-stage and low-grade cancers, therefore a combination of cytology and measurements of the ADAM12 level could increase the sensitivity to almost 100%.

To further validate the sensitivity, a larger sample size needs to be examined, and a larger study of patients with non-neoplastic bladder disorders should be included to predict the specificity of the assay. This study thus adds to our recent study on breast cancer, in which the present inventors reported that increased urinary levels of ADAM12 were found to correlate with breast cancer progression. In fact, the present inventors found that the “strength” (i.e., with regard to sensitivity, accuracy, and false-negative ratios) of ADAM12 in differentiating patients with breast cancer from those without was comparable to a number of other tumor markers currently in clinical use. Together, these two studies strongly advocate for further studies to determine the efficacy of urinary ADAM12 level as a routine biomarker for the prediction, diagnosis, and monitoring of progression of disease.

The present inventors here shows that ADAM12 is present in increased amounts in urine from bladder cancer patients when compared with the levels found in the urine of healthy controls.

In the present study, the present inventors were able to detect ADAM12 in the urine from all healthy individuals tested, whereas previously it was found that ADAM12 was only detected in about 15% of control samples. The difference in detection rate could be related to differences in sampling and storage of the specimens or the membranes used for electrophoretic transfers in the two studies.

The present inventors have found that Immobilon-P (PDVF) membranes more sensitive than nitrocellulose. The present inventors investigated which cells in the bladder might produce ADAM12 found in the normal urine.

Immunohistochemistry analysis of normal urothelium with an antibody that recognizes both ADAM12-L and ADAM12-S demonstrated that the umbrella cells, the outer layer of specialized cells, exhibited strong immunostaining, while the underlying epithelium stained more weakly.

The present inventors therefore suggest that the normal urothelium represents the most likely source of ADAM12 in normal urine. The identity of ADAM12 in urine was validated using a number of different domain-specific antibodies. ADAM12 appears as a 68 kDa protein band representing the mature form and a 27 kDa band representing the prodomain that remains associated with the rest of the molecule following secretion. The 68 kDa band could represent the mature form of ADAM12-S, a shed or otherwise truncated form of ADAM12-L, or a mixture of the two. To examine whether ADAM12-S is present in the urine of bladder cancer patients, the present inventors examined urine using a polyclonal antibody that specifically recognizes a carboxy-terminus ADAM12-S peptide. The 68 kDa band was detected in urine from bladder cancer patients, confirming the presence of ADAM12-S. In contrast, polyclonal antibodies against the carboxy-terminus of ADAM12-L did not detect a band in bladder cancer urine, suggesting that full-length ADAM12-L or a fragment truncated at the N-terminal part is not present in significant amounts (data not shown). It is still possible, however, that ADAM12-L could be shed from cell membranes and appear in the urine as a “tailless fragment.” Both previous studies (22) and the data obtained in the present study demonstrate that the level of ADAM12 is increased in the urine of cancer patients. The present inventors hypothesize that ADAM12 is produced by the tumor cells and escapes into the urine—and may be designated “tumor ADAM12.” The present inventors also suggest that the normal urothelium produces more ADAM12 in the presence of a neighboring tumor—and may be designated “cytokine-induced ADAM12”.

Using densitometric quantitation of the 68 kDa band, the present inventors found approximately 4-10 μg ADAM12/ml urine in cancer urine.

In normal urine, ADAM12 was only weakly detected ie less than 1 μg/ml urine (FIG. 5D,E).

Thus in one embodiment, the present invention relates to any of the methods disclosed herein, wherein the sample obtained and used is a urine sample.

### Specificity and Sensitivity

The present invention relates to methods for determining whether an individual is likely to have cancer, comprising determining the ADAM8, ADAM10 and/or the ADAM12 level in a sample and indicating the individual as having a high likelihood of having cancer if the parameter is at or beyond a discriminating value and indicating the individual as unlikely of having cancer if the parameter is not at or beyond the discriminating value.

The discriminating value is a value which has been determined by measuring the parameter in both a healthy control population and a population with known cancer thereby determining the discriminating value which identifies the cancer population with either a predetermined specificity or a predetermined sensitivity based on an analysis of the relation between the parameter values and the known clinical data of the healthy control population and the cancer population. The discriminating value determined in this manner is valid for the same experimental setup in future individual tests.

Thus, in one embodiment, the present invention relates to a method as described herein, wherein the reference level is predetermined.

The sensitivity of any given diagnostic test define the proportion of individuals with a positive response who are correctly identified or diagnosed by the test, e.g. the sensitivity is 100%, if all individuals with a given condition have a positive test. The specificity of a given screening test reflects the proportion of individuals without the condition who are correctly identified or diagnosed by the test, e.g. 100% specificity is, if all individuals without the condition have a negative test result.

Sensitivity is defined as the proportion of individuals with a given condition, who are correctly identified by the described methods of the invention.

Specificity herein is defined as the proportion of individuals without the condition, who are correctly identified by the described methods of the invention.

Thus in one embodiment, the present invention relates to a method for screening an individual for bladder cancer said method comprising the steps of:
a) determining the level of ADAM12 in a sample obtained from the individual,
b) constructing a percentile plot of said level of ADAM12 obtained from a healthy population,
c) constructing a ROC (receiver operating characteristics) curve based on the ADAM12 level determined in the healthy population and on the ADAM12 level determined in a population who has developed bladder cancer,
d) selecting a desired specificity,
e) determining from the ROC curve the sensitivity corresponding to the desired specificity,
f) determining from the percentile plot the ADAM12 level corresponding to the determined sensitivity; and
g) predicting the individual to have bladder cancer, if the ADAM12 level in the sample is equal to or higher than said ADAM12 level corresponding to the determined specificity and predicting the individual as unlikely or not to having bladder cancer if the ADAM12 level in the sample is lower than said ADAM12 level corresponding to the determined specificity.

In another embodiment, the present invention relates to a method for screening an individual for bladder cancer said method comprising the steps of:
a) determining the level of ADAM12 in a sample obtained from the individual,
b) constructing a percentile plot of said level of ADAM12 obtained from a healthy population,
c) selecting a desired sensitivity,
d) determining from the percentile plot the ADAM12 level corresponding to the desired sensitivity; and
e) predicting the individual to have bladder cancer, if the ADAM12 level in the sample is equal to or higher than said ADAM12 level corresponding to the determined specificity and predicting the individual as unlikely or not to having bladder cancer if the ADAM12 level in the sample is lower than said ADAM12 level corresponding to the determined specificity.

Again it should be understood that any feature and/or aspect discussed above in connection with the methods of ADAM12 according to the invention apply by analogy to the ADAM8 and/or ADAM10 according to the invention.

### Receiver-Operating Characteristics

Accuracy of a diagnostic test is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision threshold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease, latent or recent infection versus no infection, or benign versus malignant disease.

In each case, the ROC plot depicts the overlap between the two distributions by plotting the sensitivity versus 1—specificity for the complete range of decision thresholds. On the y-axis is sensitivity, or the true-positive fraction [defined as (number of true-positive test results) (number of true-positive+number of false-negative test results]. This has also been referred to as positivity in the presence of a disease or condition. It is calculated solely from the affected subgroup. On the x axis is the false-positive fraction, or 1—specificity [defined as (number of false-positive results)/(number of true-negative+number of false-positive results)]. It is an index of specificity and is calculated entirely from the unaffected subgroup.

Because the true- and false-positive fractions are calculated entirely separately, by using the test results from two different subgroups, the ROC plot is independent of the prevalence of disease in the sample. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 450 diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. The most common global measure is the area under the ROC plot. By convention, this area is always ≧0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

Clinical utility of the markers described herein may be assessed in comparison to and in combination with other diagnostic tools for the given conditions.

Thus, it is an object of preferred embodiments of the present invention to provide a method for the detection of bladder cancer in a mammal, wherein said method comprises the steps of:
a) determining the level of ADAM12 in said sample by detecting
   1) ADAM12 polypeptide and/or
   2) a polynucleotide coding for ADAM12 expression, and/or
   3) specific ADAM12 protease activity, preferably by detecting cleavage of IGFBP-3, a derivative thereof, or any other suitable substrate for ADAM12
b) constructing a percentile plot of said level of ADAM12 obtained from a healthy population
c) constructing a ROC (receiver operating characteristics) curve based on the ADAM12 level determined in the healthy population and on the ADAM12 level determined in a population who has developed bladder cancer
d) selecting a desired specificity
e) determining from the ROC curve the sensitivity corresponding to the desired specificity
f) determining from the percentile plot the ADAM12 level corresponding to the determined sensitivity; and
g) predicting the mammal to have bladder cancer, if the ADAM12 level in the sample is equal to or higher than said ADAM12 level corresponding to the determined specificity and predicting the mammal as unlikely or not to having bladder cancer if the ADAM12 level in the sample is lower than said ADAM12 level corresponding to the determined specificity.

The specificity of the method according to the present invention may be from 70% to 100%, more preferably 80% to 100%, more preferably 90% to 100%. Thus in one embodiment of the present invention the specificity of the invention is 80%, 81%, 82%, 83%, 84%, 85%, 86%, 87%, 88%, 89%, 90%, 91%, 92%, 93%, 94%, 95%, 96%, 97%, 98%, 99% or 100%.

The sensitivity of the method according to the present invention may be from 70% to 100%, more preferably 80% to 100%, more preferably 90% to 100%. Thus in one embodiment of the present invention the sensitivity of the invention is 80%, 81%, 82%, 83%, 84%, 85%, 86%, 87%, 88%, 89%, 90%, 91%, 92%, 93%, 94%, 95%, 96%, 97%, 98%, 99% or 100%.

The level of ADAM12 is compared to a set of reference data or a reference value such as the cut off value to determine whether the subject is at an increased risk or likelihood of e.g. bladder cancer.

To increase detection efficiency the method is further combined with at least one clinical data described below as an extra set of reference data to determine whether the subject is likely to have bladder cancer.

To determine whether the mammal is at increased risk of bladder cancer, a cut-off must be established. This cut-off may be established by the laboratory, the physician or on a case by case basis by each patient.

Alternatively cut point can be determined as the mean, median or geometric mean of the negative control group (e.g. not affected, healthy unexposed)+/−one or more standard deviations.

Cut off points can vary based on specific conditions of the individual tested such as but not limited to the risk of having the disease, occupation, geographic residence or exposure.

Cut off points can vary based on specific conditions of the individual tested such as but not limited to age, sex, genetic background, acquired or inherited compromised immune function. Doing adjustment of decision or cut off limit will thus determine the test sensitivity for detecting bladder cancer, if present, or its specificity for excluding bladder cancer if below this limit. Then the principle is that a value above the cut off point indicates an increased risk and a value below the cut off point indicates a reduced risk.

In addition test samples with indeterminate results must be interpreted separately. Indeterminate results are defined as result with an unexpectedly low level of CCL8 in the mitogen stimulated sample (PHA). The final cut point for an indeterminate CCL8 results may be decided according to the study group, especially in immunosuppressed the cut off level may be selected at a lower level.

### Cut Off Levels

As will be generally understood by those of skill in the art, methods for screening for bladder cancer are processes of decision making by comparison. For any decision making process, reference values based on subjects having the disease or condition of interest and/or subjects not having the disease or condition of interest are needed.

The cut off level (or the cut off point) can be based on several criteria including the number of subjects who would go on for further invasive diagnostic testing, the average risk of having and/or developing e.g. bladder cancer to all the subjects who go on for further diagnostic testing, a decision that any subject whose patient specific risk is greater than a certain risk level such as e.g. 1 in 400 or 1:250 (as defined by the screening organization or the individual subject) should go on for further invasive diagnostic testing or other criteria known to those skilled in the art.

The cut-off level can be adjusted based on several criteria such as but not restricted to group of individual tested. E.g. the cut off level may be lower in individuals with immunodeficiency and in patients at great risk of bladder cancer, cut off may be higher in groups of otherwise healthy individuals with low risk of developing bladder cancer.

In one embodiment the present invention discloses a method for determining if a subject has or will develop bladder cancer, which comprises:
(a) obtaining from the subject a sample, and
(b) quantitatively determining the concentration of the markers presented herein present in the sample, the presence of the markers present in the sample at a concentration equal to or higher and/or lower than the selected cut off indicating that the subject is likely to have bladder cancer.

The discriminating value is a value which has been determined by measuring the parameter in both a healthy control population and a population, as described above.

In the specific experimental setups described herein the level threshold of ADAM12 useful as a cut off value was found to be in the range of but not limited to 14 pg/ml to 1000 pg/ml. Preferably the cut off value may be 1 pg/ml, 2 pg/ml, 3 pg/ml, 4 pg/ml, 5 pg/ml, 6 pg/ml, 7 pg/ml, 8 pg/ml, 9 pg/ml, 10 pg/ml, 11 pg/ml, 12 pg/ml, 13 pg/ml, 14 pg/ml, or 15 pg/ml.

Dilution of sample or other parameters will result in other values, which can be determined in accordance with the teachings herein. Other experimental setups and other parameters will result in other values, which can be determined in accordance with the teachings herein.

### Large Group Screening

The cut off level can be different, if a single patient with symptoms has to be diagnosed or the test is to be used in a screening of a large number of individuals in a population.

Although any of the known analytical methods for measuring the levels of these analytes will function in the present invention, as obvious to one skilled in the art, the analytical method used for each marker must be the same method used to generate the reference data for the particular marker. If a new analytical method is used for a particular marker, a new set of reference data, based on data developed with the method, must be generated.

### Statistics

The multivariate DISCRIMINANT analysis and other risk assessments can be performed on the commercially available computer program statistical package Statistical Analysis System (manufactured and sold by SAS Institute Inc.) or by other methods of multivariate statistical analysis or other statistical software packages or screening software known to those skilled in the art.

As obvious to one skilled in the art, in any of the embodiments discussed above, changing the risk cut-off level of a positive test or using different a priori risks which may apply to different subgroups in the population, could change the results of the discriminant analysis for each group.

A stability tests may be propose where ADAM12 is highly stable with routine handling (i.e. freezing or storage for prolonged periods of time at temperatures below 10 degrees C.); thus, the present inventors conclude that ADAM12 is an attractive analyte for clinical use. The data presented here suggest that ADAM12 is a potentially valuable marker for use in prognosis, diagnosis, monitoring and screening of bladder cancer.

### ADAMs

The ADAMs (A Disintegrin And Metalloprotease) constitute a multidomain glycoprotein family with proteolytic and cell-adhesion activities. The ADAMs, like the MMPs, belong to the superfamily of zinc-dependent metzincin proteases, and consist of more than 35 members that are multidomain transmembrane proteins with protease, cell adhesive, and signaling activities.

Thus, ADAMs may play diverse roles in different tissues. They induce ectodomain shedding of growth factors, cytokines, and their receptors, and they bind to integrins and syndecans, influencing cell-cell and cell-matrix interactions.

The prototype ADAM contains, from the N-terminus, a signal peptide, a prodomain, a metalloprotease domain, a disintegrin domain, a cysteine-rich domain, an epidermal growth factor (EGF)-like domain, a transmembrane domain, and a cytoplasmic domain. Four ADAMs (ADAM9, 11, 12, and 28) exist in alternatively spliced secreted (-S) forms that do not contain transmembrane and cytoplasmic domains.

### Different Expression

As used herein, the term “differential expression” refers to a difference in the level of expression of the RNA and/or protein products ADAM 12 possibly in combination with one or more combinatorial biomarkers of the invention, as measured by the amount or level of RNA or protein.

In reference to RNA, it can include difference in the level of expression of mRNA, and/or one or more spliced variants of mRNA of the biomarker in one sample as compared with the level of expression of the same one or more biomarkers of the invention as measured by the amount or level of RNA, including mRNA and/or one or more spliced variants of mRNA in a second sample. “Differentially expressed” or “differential expression” can also include a measurement of the protein, or one or more protein variants encoded by the biomarker of the invention in a sample or population of samples as compared with the amount or level of protein expression, including one or more protein variants of the biomarker or biomarkers of the invention. Differential expression can be determined as described herein and as would be understood by a person skilled in the art. The term “differentially expressed” or “changes in the level of expression” refers to an increase or decrease in the measurable expression level of a given product of the biomarker as measured by the amount of RNA and/or the amount of protein in a sample as compared with the measurable expression level of a given product of the biomarker in a second sample. The first sample and second sample need not be from different patients, but can be samples from the same patient taken at different time points. The term “differentially expressed” or “changes in the level of expression” can also refer to an increase or decrease in the measurable expression level of a given biomarker in a population of samples as compared with the measurable expression level of a biomarker in a second population of samples. As used herein, “differentially expressed” when referring to a single sample can be measured using the ratio of the level of expression of a given biomarker in said sample as compared with the mean expression level of the given biomarker of a control population wherein the ratio is not equal to 1.0.

Differentially expressed can also be used to include comparing a first population of samples as compared with a second population of samples or a single sample to a population of samples using either a ratio of the level of expression or using p-value. When using p-value, a measure of the statistical significance of the differential expression, a nucleic acid transcript including hnRNA and mRNA is identified as being differentially expressed as between a first and second population when the p-value of less than 0.3, 0.2, 0.1, less than 0.05, less than 0.01, less than 0.005, less than 0.001 etc. are considered statistically significant. When determining differential expression on the basis of the ratio of the level of gene product expression, an RNA or protein gene product is differentially expressed if the ratio of the level of its RNA or protein product in a first sample as compared with that in a second sample is greater than or less than 1.0. For instance, a ratio of greater than 15 for example 1.2, 1.5, 1.7, 2, 3, 4, 10, 20, or a ratio of less than 1, for example 0.8, 0.6, 0.4, 0.2, 0.1. 0.05, of RNA or protein product of a gene would be indicative of differential expression. In another embodiment of the invention, a nucleic acid transcript including hnRNA and mRNA is differentially expressed if the ratio of the mean level of expression of a first transcript in a nucleic acid population as compared with its mean level of expression in a second population is greater than or less than 1.0. For instance, a ratio of greater than 1, for example 1.2, 1.5, 1.7, 2, 3, 4, 10, 20, or a ratio less than 1, for example 0.8, 0.6, 0.4, 0.2, 0.1. 0.05 would be indicative of differential expression.

In another embodiment of the invention a nucleic acid transcript including hnRNA, and mRNA is differentially expressed if the ratio of its level of expression in a first sample as compared with the mean of the second population is greater than or less than 1.0 and includes for example, a ratio of greater than 1, for instance 1.2, 1.5, 1.7, 2, 3, 4, 10, 20, or a ratio less than l, for example 0.8, 0.6, 0.4, 0.2, 0.1. 0.05. “Differentially increased expression” refers to 1.1 fold, 1.2 fold, 1.4 fold, 1.6 fold, 1.8 fold, or more, relative to a standard, such as the mean of the expression level of the second population. “Differentially decreased expression” refers to less than 1.0 fold, 0.8 fold, 0.6 fold, 0.4 fold, 0.2 fold, 0.1 fold or less, relative to a standard, such as the mean of the expression level of the second population.

### Grading

The stage of a cancer tells the skilled person how far the cancer has spread. It is important because treatment is often decided according to the stage of a cancer. There are different ways of staging cancers. The most common is the TNM system. This is common to all cancers. TNM stands for ‘tumour, node, metastasis’.

So this staging system takes into account how deep the tumour has grown into the bladder, whether there is cancer in the lymph nodes and whether the cancer has spread to any other part of the body. The TNM system is a quick and detailed way of writing down the stage of a cancer accurately.

Another way of staging cancers is number staging. This is used for other cancers, but not so much for bladder cancer. There are usually 4 main stages. Stage 1 is the earliest cancer and stage 4 the most advanced. With bladder cancer, it is more usual to refer to early (or superficial) bladder cancer, invasive bladder cancer and advanced bladder cancer.

Cancer grade means how well developed the cell looks like under the microscope.

The more the cancer cell looks like a normal cell, the more it will behave like one

Cancer cells are usually classed as low, medium or high grade. Other may talk about grades 1, 2, or 3, where G1 is low grade. A low grade cancer is likely to be less aggressive in its behaviour than a high grade one. One cannot be certain how the cells will behave, but grade is a useful indicator.

The present invention is able to distinguish between the various grades of bladder cancer such as but no limited to the ‘T’ stages of bladder cancer.

The ‘T’ of TNM tells you how far into the bladder the cancer cells have grown:
CIS—very early cancer cells are detected only in the innermost layer of the bladder lining
Ta—the cancer is just in the innermost layer of the bladder lining
T1—the cancer has started to grow into the connective tissue beneath the bladder lining
T2—the cancer has grown through the connective tissue into the muscle
T2a—the cancer has grown into the superficial muscle
T2b—the cancer has grown into the deeper muscle
T3—the cancer has grown through the muscle into the fat layer
T3a—the cancer in the fat layer can only be seen under a microscope (microscopic invasion)
T3b—the cancer in the fat layer can be seen on tests, or felt by the doctor (macroscopic invasion)
T4—the cancer has spread outside the bladder
T4a—the cancer has spread to the prostate, womb or vagina
T4b—the cancer has spread to the wall of the pelvis and abdomen

The present invention is also able to distinguish between the various grades of bladder cancer such as but no limited to the ‘N’ stages of bladder cancer. There are four lymph node stages in bladder cancer. These relate to lymph nodes in the pelvis (the lower part of your tummy, inside your hip bones, or pelvic girdle). The stages are:
N0—no cancer in any lymph nodes
N1—one affected lymph node smaller than 2 cm across
N2—one affected lymph node larger than 2 cm, but smaller than 5 cm. Or more than one node affected, but all of them smaller than 5 cm across
N3—at least one affected lymph node larger than 5 cm across

The size of the lymph nodes is used because the more cancer there is growing in a lymph node, the larger it will be.

The present invention is also able to distinguish between the various grades of bladder cancer such as but no limited to the M stages of bladder cancer.

The ‘M’ stages of bladder cancer of bladder cancer are, as with most cancers, there are two stages for cancer spread or metastases. Either the cancer has spread to another body organ (M1) or it hasn't (M0).

Early bladder cancer is also called ‘superficial bladder cancer’. This includes Ta tumours, T1 tumours and carcinoma in situ (CIS). CIS is called T is in the bladder cancer TNM staging.

In one embodiment the present invention relates to a method as described, wherein said method can differentiate between different grades and stages of bladder cancer.

### Post Treatment

The present invention describes with a desired certainty whether an individual does or does not have recurrent bladder cancer. The present invention can be used to determine individuals with high likelihood to have such conditions, additional follow-up medical procedures may be recommended to determine if the individual in fact has the condition.

The differentially expressed ADAM genes identified herein also allow for the course of treatment of bladder cancer to be monitored. The present inventors e.g. found that in most of these bladder cancer cases the level of ADAM12 in the urine decreased following surgery, was minimal during the tumor-free period, but then increased again upon recurrence of tumor

In this sense, a test cell population is provided from a subject undergoing treatment for bladder cancer. If desired, test cell populations are obtained from the subject at various time points, before, during, and/or after treatment. Expression of one or more of the ADAM genes in the test cell population is then determined and compared to expression of the same genes in a reference cell population which includes cells whose bladder cancer state is known. In the context of the present invention, the reference cells have not been exposed to the treatment of interest.

If the reference cell population contains no bladder cancer cells, a similarity in the expression of an ADAM gene in the test cell population and the reference cell population indicates that the treatment of interest is efficacious. However, a difference in the expression of an ADAM gene in the test cell population and a normal control reference cell population indicates a less favorable clinical outcome or prognosis. Similarly, if the reference cell population contains bladder cancer cells, a difference between the expression of an ADAM gene in the test cell population and the reference cell population indicates that the treatment of interest is efficacious, while a similarity in the expression of an ADAM gene in the test population and a bladder cancer control reference cell population indicates a less favorable clinical outcome or prognosis.

Additionally, the expression level of one or more ADAM genes determined in a biological sample from a subject obtained after treatment {i.e., post-treatment levels) can be compared to the expression level of the one or more ADAM genes determined in a biological sample from a subject obtained prior to treatment onset (i.e., pre-treatment levels).

If the ADAM gene is an up-regulated gene, a decrease in the expression level in a post-treatment sample indicates that the treatment of interest is efficacious while an increase or maintenance in the expression level in the post-treatment sample indicates a less favorable clinical outcome or prognosis.

Conversely, if the ADAM gene is a down-regulated gene, an increase in the expression level in a post-treatment sample can indicate that the treatment of interest is efficacious while a decrease or maintenance in the expression level in the post-treatment sample indicates a less favorable clinical outcome or prognosis.

Thus in one embodiment, the present invention relates to a method for determining whether an individual is likely to have recurrent bladder cancer after being treated for bladder cancer, said method comprises the steps of:
a) determining the level of ADAM12 in a sample obtained post-treatment from the individual treated for bladder cancer
b) comparing said level with a reference level;
c) identifying whether the level is different from said reference level and
   evaluating whether the individual is likely to have recurrent bladder cancer, if the level is higher than the reference level.

Further, the present invention also relates to a method for determining whether an individual is likely to have recurrent bladder cancer after being treated for bladder cancer, said method comprises the steps of:
a) determining the level of ADAM12 in a sample obtained post-treatment from an individual treated for bladder cancer
b) constructing a percentile plot of said level of ADAM12 obtained from a healthy population
c) constructing a ROC (receiver operating characteristics) curve based on the ADAM12 level determined in the healthy population and on the ADAM12 level determined in a population with known recurrent bladder cancer
d) selecting a desired specificity
e) determining from the ROC curve the sensitivity corresponding to the desired specificity
f) determining from the percentile plot the ADAM12 level corresponding to the determined sensitivity; and
g) indicating the individual to have bladder cancer, if the post-treatment ADAM12 level in the sample is equal to or higher than said ADAM12 level corresponding to the determined specificity and predicting the individual as unlikely or not to having recurrent bladder cancer if the ADAM12 level in the sample is lower than said ADAM12 level corresponding to the determined specificity.

As used herein, the term “efficacious” indicates that the treatment leads to a reduction in the expression of a pathologically up-regulated gene, an increase in the expression of a pathologically down-regulated gene or a decrease in size, prevalence, or metastatic potential of the cancer in a subject.

When a treatment of interest is applied prophylactically, the term “efficacious” means that the treatment retards or prevents a bladder cancer tumor from forming or retards, prevents, or alleviates a symptom of clinical bladder cancer. Assessment of bladder cancer tumors can be made using standard clinical protocols.

In addition, efficaciousness can be determined in association with any known method for diagnosing or treating bladder cancer. Bladder cancer can be diagnosed, for example, by identifying symptomatic anomalies, e.g., weight loss, abdominal pain, back pain, anorexia, nausea, vomiting and—generalized malaise, weakness, and jaundice.

It is known in the art that the level of any disease-specific molecular marker may increase as a response to e.g. surgical operation performed to remove the primary tumor. Accordingly, a sample taken shortly after the surgery may have e.g. an elevated ADAM12 level irrespectively of the presence or absence of recurrent colorectal cancer, simply due to the post-treatment trauma and stress. Given this knowledge, a skilled practioner will select a suitable timing for initiating the monitoring of e.g. ADAM12 shortly after the treatment. Nonetheless, any moment may be selected.

Based on this knowledge, a skilled practitioner will initiate the monitoring of the ADAM12 level e.g. 3 months after the treatment. If the ADAM12 level decreases to below the pre-determined ADAM12 level at any time between 3 months after surgery and e.g. 6 months after surgery and persistently stays below the pre-determined level, the individual will be likely not to have recurrent bladder cancer. However, if the ADAM12 level remains at substantially the same level or even increases after treatment such as but not limited to removal of the tumor as was measured before surgery, the patient is likely to have recurrent bladder cancer.

The 3 month period post-treatment before taking the sample to determine the ADAM12 level is similar to recommendations for the use of CEA as recurrent cancer marker. According to these recommendations samples are taken in three months intervals after the treatment during the first year and in six months intervals thereafter.

### Marker

As used herein, the term “marker” or “biomarker” refers to a gene that is differentially expressed in individuals having bladder cancer or a stage of bladder cancer as compared with those not having bladder cancer, or said stage of bladder cancer (although individuals may have other disease(s)) and can include a gene that is differentially expressed in individuals having superficial bladder cancer as compared with those not having bladder cancer.

Combination with Other Markers

In one embodiment, measuring e.g. ADAM12 in combination with one or more of combinatorial marker may reduce the number of false positive and increase the discriminatory power.

Thus in one embodiment, the present invention relates to methods as described herein, wherein the ADAM12 level is combined with values from at least one combinatorial marker, such as but not limited to ADAM8 and ADAM10. Any marker or test correlating to bladder cancer or even cancer in general known to the skilled addressee may be selected.

In one embodiment the present invention the combinatorial marker is selected from the group consisting of ADAM8, ADAM10, MMP2 and MMP9.

Bladder tumor antigen (BTA), nuclear matrix protein 22 (NMP22), fibronectin and its fragment, and cytokeratin (CK) 8, 18, 19, and 20 are among the most commonly evaluated markers, thus is included in the term “combinatorial biomarkers of the invention”, which also refers e.g. to any one or more biomarkers as disclosed in WO 06/121710 hereby expressly incorporated by reference in its entirety.

Commonly used test is the ImmunoCyt test. This is another test for cancer-related substances in the urine and may be more sensitive than cytology for certain cancers. Other tests include the BTA stat test, and the UroVysion test which looks at the DNA of the cells in bladder washings. Some doctors find these tests useful, but most feel more research is needed before they should be used routinely. But with the addition of e.g. ADAM12 to such tests high discriminatory power is obtained.

Combination with Cytology

The discriminatory power may also be enhanced by combining the level of e.g ADAM12 with the other clinical and cytological characteristics of bladder cancer.

In most cases, blood in the urine (hematuria) is the first warning signal of bladder cancer. Sometimes, there is enough blood to color the urine. Depending on the amount of blood, the urine may be very pale yellow-red or, less often, darker red.

In other cases, the color of the urine is normal but small amounts of blood can be found by urine tests done because of other symptoms or as part of a general medical check-up.

Blood in the urine is not a sure sign of bladder cancer. It may also be caused by infections of the kidneys, bladder, or urethra, other benign kidney diseases, benign tumors of the kidney, bladder or ureter, and kidney or bladder stones. Blood may be present one day and absent the next, with the urine remaining clear for weeks or months. With bladder cancer, blood eventually reappears. Usually the early stages of bladder cancer cause bleeding but little or no pain.

Change in bladder habits or irratative symptoms: Having to urinate more often than usual or having a feeling of needing to go but not being able to is also a symptom of bladder cancer. Rarely, people with bladder cancer notice burning during urination.

If bladder cancer is suspected, doctors will recommend a cystoscopy. A cystoscope is a slender tube with a lens and a light. It is placed into the bladder through the urethra. It permits the doctor to view the inside of the bladder. This can be done in the office by a urologist, a specialist in diseases of the urinary system. Usually the first cystoscopy will be with a small flexible fiberoptic device. Some sort of local anesthesia is used such as an anesthetic gel, but it can be general or spinal. If suspicious areas or growths are seen, a small piece of tissue is removed and examined (biopsy). Also at this time washings will be done for cytology.

Fluorescence cystoscopy may be used at the time of cystoscopy by e.g use of porphyrins.

Urine cytology: The urine is examined under a microscope to look for cancerous or precancerous cells. Cytology will also be done on bladder washings taken at the time of cystoscopy. Bladder washing samples are taken by placing a salt solution into the bladder through a catheter and then removing the solution for microscopic testing. If the test does not find cancer, this doesn't mean there isn't any there. The test can sometimes fail to find cancer.

Urine culture: A urine culture is done to rule out an infection. Infections and bladder cancers can sometimes cause similar symptoms. A sample of urine is tested in the lab to see if bacteria are present. It may take 48 to 72 hours to get the results of this test.

Biopsy: A sample of bladder tissue is removed from a suspicious area or growth, using instruments operated through the cystoscope. The sample is examined under the microscope by a pathologist. The biopsy procedure can identify bladder cancers and tell what type of cancer (urothelial carcinoma, squamous cell carcinoma, adenocarcinoma, etc.) is present. It can also tell how deeply the cancer has penetrated.

Imaging test such as Intravenous pyelogram (IVP), Retrograde pyelography, Chest x-ray, Computed tomography (CT), Magnetic resonance imaging (MRI) scans, Ultrasound, Bone scans, and Positron Emission Tomography (PET) scans may be combined with the markers of the present invention.

### Theranostic

The term theranostics describes the use of diagnostic testing to diagnose the disease, choose the correct treatment regime and monitor the patient response to therapy.

In traditional medical practice therapeutic choices follow diagnosis, which may be based on clinical signs alone, or may be made in conjunction with an in vivo or in vitro diagnostic test. However, the effectiveness of the prescribed drug therapy and the likelihood of side effects often cannot be predicted for individual patients.

Theranostics (therapy specific diagnostics) are being developed specifically for predicting and assessing drug response in individual patients rather than diagnosing disease.

Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay.

Theranostics holds the key to improving the success rate of drug candidates entering clinical trials (currently around 20%) and to marketing approved drugs more effectively.

Future progress in theranostics will draw on developments in pharmacogenomics, which seeks to establish correlations between responses to specific drugs and the genetic profiles of patients. The most common form of genetic profiling relies on the use of DNA sequence variations called single nucleotide polymorphisms (SNPs). Currently patient genetic data is used mainly to make drug development more efficient and cost-effective. SNP genotyping is being used to determine genotypes associated with drug responsiveness, side effects, or optimal dose. Nova Molecular pioneered SNP genotyping of the apolipoprotein E gene to predict Alzheimer's disease patients' responses to cholinomimetic therapies and it is now widely used in clinical trials of new drugs for this indication. Stratifying patients according to variables that may be predictors of safety or efficacy can enhance the statistical power of a clinical trial.

DNA microarray technologies are being used increasingly to evaluate patient-to-patient variations in both gene sequence and gene expression.

Personalized medicine is the use of detailed information about a patient's genotype or level of gene expression and a patient's clinical data in order to select a medication, therapy or preventative measure that is particularly suited to that patient at the time of administration.

The benefits of this approach are in its accuracy, efficacy, safety and speed. The term emerged in the late 1990s with progress in the Human Genome Project. Research findings over the past decade, or so, in biomedical research have unfolded a series of new, predictive sciences that share the appendage-omics (genomics, proteomics, metabolomics, cytomics). These are opening the possibility of a new approach to drug development as well as unleashing the potential of significantly more effective diagnosis, therapeutics, and patient care.

Thus in one aspect, the present invention relates to a method for treating bladder cancer comprising:
identifying a mammal expressing elevated levels of ADAM8, ADAM10 and/or ADAM12, and
   administering to said mammal an effective amount of a drug sufficient to reduce tumor growth or prevent metastasis.

Drugs commonly used to treat bladder cancer include valrubicin (Valstar®), thiotepa (Thioplex®), mitomycin, and doxorubicin (Rubex®).

### Kits

In one embodiment the present invention relates to a kit comprising a detection reagent which binds to any nucleic acid sequence of ADAM12 and/or polypeptides encoded thereby for the determination of bladder cancer.

One embodiment of the present invention relates to a kit for screening for, assessing the prognosis of an individual with bladder cancer, which comprises a reagent selected from the group consisting of:
(a) a reagent for detecting mRNA of the ADAM12 gene;
(b) a reagent for detecting the ADAM12 protein; and
(c) a reagent for detecting the biological activity of the ADAM12 protein.

In a specific embodiment of such kit reagent is an antibody against the ADAM12 protein.

The present invention provides kits for measuring the expression of the protein and RNA products of ADAM12 in combination with at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, all or any combinational biomarkers mentioned herein.

Such kits comprise materials and reagents required for measuring the expression of such protein and RNA products. In specific embodiments, the kits may further comprise one or more additional reagents employed in the various methods, such as: (1) reagents for stabilizing and/or purifying RNA from the sample (2) primers for generating test nucleic acids; (3) dNTPs and/or rNTPs (either premixed or separate), optionally with one or more uniquely labelled dNTPs and/or rNTPs (e.g., biotinylated or Cy3 or Cy5 tagged dNTPs); (4) post synthesis labelling reagents, such as chemically active derivatives of fluorescent dyes; (5) enzymes, such as reverse transcriptases, DNA polymerases, and the like; (6) various buffer mediums, e.g., reaction, hybridization and washing buffers; (7) labelled probe purification reagents and components, like spin columns, etc.; and (8) protein purification reagents; (9) signal generation and detection reagents, e.g., streptavidin-alkaline phosphatase conjugate, chemifluorescent or chemiluminescent substrate, and the like.

In particular embodiments, the kits comprise prelabeled quality controlled protein and or RNA isolated from a sample (e.g., blood or chondrocytes or synovial fluid) for use as a control.

In some embodiments, the kits are RT-PCR or qRT-PCR kits.

In other embodiments, the kits are nucleic acid arrays and protein arrays. Such kits according to the subject invention will at least comprise an array having associated protein or nucleic acid members of the invention and packaging means therefore. Alternatively the protein or nucleic acid members of the invention may be pre-packaged onto an array.

In some embodiments, the kits are Quantitative RT-PCR kits. In one embodiment, the quantitative RT-PCR kit includes the following: (a) primers used to amplify each of a combination of biomarkers of the invention; (b) buffers and enzymes including an reverse transcriptase; (c) one or more thermos table polymerases; and (d) Sybr® Green. In another embodiment, the kit of the invention also includes (a) a reference control RNA and (b) a spiked control RNA.

The invention provides kits that are useful for (a) diagnosing individuals as having bladder cancer and/or early stage bladder cancer. For example, in a particular embodiment of the invention a kit is comprised a forward and reverse primer wherein the forward and reverse primer are designed to quantitate expression of all of the species of mRNA corresponding to each of the biomarkers as identified in accordance with the invention useful in determining whether an individual has bladder cancer and/or early stage bladder cancer or not. In certain embodiments, at least one of the primers is designed to span an exon junction.

The invention provides kits that are useful for detecting, diagnosing, monitoring and prognosing bladder cancer based upon the expression of protein or RNA products of ADAM12 possibly in combination with at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, all or any combination of the combinatorial biomarkers of the invention in a sample.

In certain embodiments, such kits do not include the materials and reagents for measuring the expression of a protein or RNA product of a biomarker of the invention that has been suggested by the prior art to be associated with bladder cancer, hi other embodiments, such kits include the materials and reagents for measuring the expression of a protein or RNA product of a combinatorial biomarker of the invention that has been suggested by the prior art to be associated with bladder cancer and at least 1, at least 2, at least 3, at least 4, at least 5, at least 6, at least 7, at least 8, at least 9, at least 10, at least 15, at least 20, at least 25, at least 30, at least 35, at least 40, at least 45 or more genes other than the combinatorial biomarkers of the invention.

The invention provides kits useful for monitoring the efficacy of one or more therapies that a subject is undergoing based upon the expression of a protein or RNA product of ADAM12 in combination with any number of up to at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, all or any combination of the combinatorial biomarkers of the invention in a sample. In certain embodiments, such kits do not include the materials and reagents for measuring the expression of a protein or RNA product of a biomarker of the invention that has been suggested by the prior art to be associated with bladder cancer. In other embodiments, such kits include the materials and reagents for measuring the expression of a protein or RNA product of ADAM12 together with a combinatorial biomarker of the invention that has been suggested by the prior art to be associated with bladder cancer and any number of up to at least 1, at least 2, at least 3, at least 4, at least 5, at least 6, at least 7, at least 8, at least 9, at least 10, at least 15, at least 20, at least 25, at least 30, at least 35, at least 40, at least 45 or more genes other than the combinatorial biomarkers of the invention.

The invention provides kits using for determining whether a subject will be responsive to a therapy based upon the expression of a protein or RNA product of ADAM12 possibly in combination with any number of up to at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, all or any combination of the combinatorial biomarkers of the invention in a sample.

In a specific embodiment, such kits comprise materials and reagents that are necessary for measuring the expression of a RNA product of a biomarker of the invention. For example, a microarray or RT-PCR kit.

For nucleic acid microarray kits, the kits generally comprise probes attached to a solid support surface. The probes may be labelled with a detectable label. In a specific embodiment, the probes are specific for an exon(s), an intron(s), an exon junction(s), or an exon-intron junction(s)), of RNA products of ADAM12 possibly in combination with any number of up to 1, 2, 3, 4, 5, 6, 7, 8, 9, 10, 15, 20, 25, 30, 35, 40, 45, 50, all or any combination of the combinatorial biomarkers of the invention.

The microarray kits may comprise instructions for performing the assay and methods for interpreting and analyzing the data resulting from the performance of the assay. In a specific embodiment, the kits comprise instructions for diagnosing bladder cancer. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own a suitable container.

For RT-PCR kits, the kits generally comprise pre-selected primers specific for particular RNA products (e.g., an exon(s), an intron(s), an exon junction(s), and an exon-intron junction(s)) of ADAM12 possibly in combination with any number of up to 1, 2, 3, 4, 5, 6, 7, 8, 9, 10, 15, 20, 25, 30, 35, 40, 45, 50, all or any combination of the combinatorial biomarkers of the invention. The RT-PCR kits may also comprise enzymes suitable for reverse transcribing and/or amplifying nucleic acids (e.g., polymerases such as Taq), and deoxynucleotides and buffers needed for the reaction mixture for reverse transcription and amplification. The RT-PCR kits may also comprise probes specific for RNA products of ADAM12 and possibly any number of up to 1, 2, 3, 4, 5, 6, 7, 8, 9, 10, 15, 20, 25, 30, 35, 40, 45, 50, all or any combination of the combinatorial biomarkers of the invention. The probes may or may not be labelled with a detectable label (e.g., a fluorescent label). Each component of the RT-PCR kit is generally in its own suitable container. Thus, these kits generally comprise distinct containers suitable for each individual reagent, enzyme, primer and probe. Further, the RT-PCR kits may comprise instructions for performing the assay and methods for interpreting and analyzing the data resulting from the performance of the assay. In a specific embodiment, the kits contain instructions for diagnosing bladder cancer.

In a specific embodiment, the kit is a real-time RT-PCR kit. Such a kit may comprise a 96 well plate and reagents and materials necessary for e.g. SYBR Green detection. The kit may comprise reagents and materials so that beta-actin can be used to normalize the results. The kit may also comprise controls such as water, phosphate buffered saline, and phage MS2 RNA. Further, the kit may comprise instructions for performing the assay and methods for interpreting and analyzing the date resulting from the performance of the assay. In a specific embodiment, the instructions state that the level of a RNA product of ADAM12 AND any number of up to 1, 2, 3, 4, 5, 6, 7, 8, 9, 10, 15, 20, 25, 30, 35, 40, 45, 50, all or any combination of the combinatorial biomarkers of the invention should be examined at two concentrations that differ by, e.g., 5 fold to 10-fold.

For antibody based kits, the kit can comprise, for example: (1) a first antibody (which may or may not be attached to a solid support) which binds to ADAM12 and any combinatorial protein of interest (e.g., a protein product of any number of up to 1, 2, 3, 4, 5, 6, 7, 8, 9, 10, 15, 20, 25, 30, 35, 40, 45, 50, all or any combination of the combinatorial biomarkers of the invention); and, optionally, (2) a second, different antibody which binds to either the protein, or the first antibody and is conjugated to a detectable label (e.g., a fluorescent label, radioactive isotope or enzyme). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analyzing the data resulting from the performance of the assay.

In a specific embodiment, the kits contain instructions for diagnosing bladder cancer.

### Reference

In order to determine the clinical severity of bladder cancer, means for evaluating the detectable signal of the present markers measured involves a reference or reference means.

The reference also makes it possible to count in assay and method variations, kit variations, handling variations and other variations not related directly or indirectly to the various ADAM12 levels.

In the context of the present invention, the term “reference” relates to a standard in relation to quantity, quality or type, against which other values or characteristics can be compared, such as e.g. a standard curve.

In one embodiment the reference level is predetermined.

The reference data reflect the level of ADAM 12 for subjects having bladder cancer (also referred to as affected, exposed, vaccinated, infected or diseased) and/or the level of ADAM12 for normal subjects (also referred to as unaffected, unexposed, un vaccinated, uninfected, or healthy).

As used herein, “normal” refers to an individual or group of individuals who have not shown any evidence of bladder cancer, or symptoms thereof including blood in urine, and have not been diagnosed with bladder cancer or the possibility that they may have bladder cancer. Preferably said “normal” refers to an individual or group of individuals who is not at an increased risk of having bladder cancer.

In addition, preferably said normal individual(s) is not on medication affecting bladder cancer and has not been diagnosed with any other disease.

More preferably normal individuals have similar sex, age as compared with the test samples. “Normal”, according to the invention, also refers to a samples isolated from normal individuals and includes total RNA or mRNA isolated from normal individuals. A sample taken from a normal individual can include RNA isolated from a tissue sample. As used herein, “nucleic acid(s)” is interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also include regions of single- or double-stranded RNA or DNA or any combinations thereof and can include expressed sequence tags (ESTs) according to some embodiments of the invention. An EST is a portion of the expressed sequence of a gene (i.e., the “tag” of a sequence), made by reverse transcribing a region of mRNA so as to make cDNA.

### Array

As defined herein, a “nucleic acid array” refers a plurality of unique nucleic acids (or “nucleic acid members”) attached to a support where each of the nucleic acid members is attached to a support in a unique pre-selected region.

In one embodiment, the nucleic acid member attached to the surface of the support is DNA.

In a preferred embodiment, the nucleic acid member attached to the surface of the support is either cDNA or oligonucleotides.

In another preferred embodiment, the nucleic acid member attached to the surface of the support is cDNA synthesised by polymerase chain reaction (PCR).

The term “nucleic acid”, as used herein, is interchangeable with the term “polynucleotide”. In another preferred embodiment, a “nucleic acid array” refers to a plurality of unique nucleic acids attached to nitrocellulose or other membranes used in Southern and/or Northern blotting techniques.

In one embodiment, a conventional nucleic acid array of ‘target’ sequences bound to the array can be representative of the entire human genome, e.g. Affymetrix chip.

In another embodiment, sequences bound to the array can be an isolated oligonucleotide, cDNA, EST or PCR product corresponding to any biomarker of the invention total cellular RNA is applied to the array.

Thus in one aspect, the present invention relates to an array comprising a nucleic acid which binds to at least one of the markers selected from the group consisting of ADAM8, ADAM10 and ADAM12 for the determination of bladder cancer.

Reference to any prior art in this specification is not, and should not be taken as, an acknowledgment or any form of suggestion that this prior art forms part of the common general knowledge in any country.

All patent and non-patent references cited in the present application, are hereby expressly incorporated by reference in their entirety.

As will be apparent, preferred features and characteristics of one aspect of the invention may be applicable to other aspects of the invention. The invention may be embodied in other specific forms without departing from the spirit or essential characteristics thereof. The foregoing embodiments are therefore to be considered in all respects illustrative rather than limiting on the invention described herein. Scope of the invention is thus indicated be the appended claims rather than by the foregoing description, and all changes that come within the meaning and range of equivalency of the claims are intended to be embraced by reference therein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The words “a”, “an”, and “the” as used herein mean “at least one” unless otherwise specifically indicated.

The invention will hereinafter be described by way of the following non-limiting Examples.

### EXAMPLES

### Example 1

This example describes in greater detail some of the materials and methods used in the experiments described herein.

### Microarray Gene Expression Profiling.

In this study, the present inventors analyzed 21 normal bladder biopsies and biopsies from 31 Ta tumors, 20 T1 tumors and 45 T2-4 tumors by microarray analysis. Bladder tumor biopsies were obtained directly from surgery after removal of the necessary amount of tissue for routine pathology examination.

Normal bladder tissue biopsies were obtained from individuals with no history of bladder tumors.

Tissue samples were frozen at −80° C. in a guanidinium thiocyanate solution for preservation of the RNA. Informed consent was obtained from all patients, and the protocols were approved by the scientific ethical committee of Aarhus County.

RNA extraction, sample labeling, hybridization to customized Affymetrix GeneChip Eos Hu03 (Affymetrix, Santa Clara, Calif., USA), and generation of expression intensity measures was performed as described by Dyrskjot et al.

Reverse transcription-polymerase chain reaction (RT-PCR) and quantitative PCR (qPCR). Total RNA was extracted and isolated as described by Dyrskjot et al. One μg RNA was treated with DNase I (Invitrogen, Carlsbad, Calif., USA) and reverse transcribed using random hexamer primers and the Transcriptor First Strand cDNA Synthesis Kit (Roche, Indianapolis, Ind., USA).

As a positive control, RNA was isolated from human rhabdomyosarcoma cells, RD (ATCC number: CCL-136, American Type Culture Collection, Manassas, Va., USA).

In addition, plasmids containing the cDNA sequence of ADAM12-L or -S were used as positive controls. Intron-spanning primers for ADAM12-L and -S were designed as follows: primers targeting ADAM12-L (forward: 5-CAGCCAAGCCTGCACTTAG-3 and reverse: 5′-AGTGAGCCGAGTTGTTCTGG-3′) produced a 101 bp fragment, and primers targeting ADAM12-S (forward: 5′-GCTTTGGAGGAAGCACAGAC-3 and reverse: 5′-TCAGTGAGGCAGTAGACGCA-3′) produced a 135 bp fragment. Primers targeting the reference gene glyceraldehyde-3-phosphate dehydrogenase (GAPDH) (forward: 5′-AAGGTCATCCCAGAGCTGAACG-3′ and reverse: 5′-TGTCATACCAGGAAATGAGC-3′) produced a 292 bp fragment. The PCR program consisted of 5 min at 95° C., followed by 35 cycles of 15 sec at 94° C., 20 sec at 55° C. (GAPDH) or 60° C. (ADAM12-L and -S), 1 min at 72° C., and a final extension step for 2 min at 72° C. Products were confirmed on a 2% agarose gel.

qPCR was performed using the LightCycler® FastStart DNA Master SYBR Green I and the LightCycler® QPCR machine (Roche). Primers targeting the reference gene 18S rRNA (forward: 5′-CGCCGCTAGAGGTGAAATTC-3′ and reverse: 5′-TTGGCAAATGCTTTCGCTC-3′) produced a 62 bp fragment (18). The qPCR program consisted of 10 min at 95° C., followed by 35 cycles of 0 sec at 95° C., 8 sec at 60° C., and 22 sec at 72° C., followed by measurement of fluorescence at 82° C. for ADAM12-L and -S for 0 sec.

The qPCR program was followed by a melting point program to check the purity of PCR products. The data were analyzed using the 2(-ΔΔC(T)) method (25). qPCR products were purified, TA cloned into pTZ57R/T (Fermentas International Inc., Burlington, Ontario, Canada), transformed into DH5a cells, and plated on Luria-Bertani (LB)-agar plates containing carbenicillin and 5-bromo-4-chloro-3-indolyl-β-[scapjd[rJ-galactopyranoside (X-Gal). Isolated plasmids were sequenced using M13 reverse (−49) primers at MWG Biotech, Ebersberg, Germany.

In Situ Hybridization for ADAM12.

Breast tumor sections from ADAM12-MMTV-PyMT and control MMTV-PyMT mice (a mouse breast cancer model) and human bladder cancer tissue arrays were used for ADAM12 mRNA in situ hybridization as described by Junker et al.

A human ADAM12 PCR product (representing nucleotides (nt) 2208 to 2397 in the cysteine-rich and EGF-like domains) was generated using full-length human ADAM12-L as a template (GenBank number AF023476). The forward primer was 5′-GGATCCAATAATACGACTCACTATAGGGAGAGGCACAAAGTGTGCAGATG-3′ containing a T7 RNA polymerase recognition site (italics) and an ADAM12 mRNA sequence (underlined) and the reverse primer was 5′-GAGAATTCATTAACCCTCACTAAAGGGAGAGTCTGTGCTTCCTCCAMGC-3 containing a T3 RNA polymerase recognition site (italics) and an ADAM12 mRNA complementary sequence (underlined).

The resulting PCR fragment was excised from a Tris-acetate (TAE) 1% seakem agarose gel (BMA product, Rockland, Me., USA) and purified by Spin-X (Costar, Cambridge, Mass., USA) as described by the manufacturer. Single-stranded sense and anti-sense ([α-35S]-UTP)— labeled RNA probes (190 bp) were generated by in vitro transcription of the purified cDNA fragment using T7 and T3 RNA polymerase (Roche). The labeled probes were purified on S-200 microspin columns (GE Healthcare Bio-Sciences AB, Uppsala, Sweden). 2×106 cpm were used per section. Paraffin sections were deparaffinized and treated with 1.25 μg/ml proteinase K for 5 min (mouse sections) or 5 μg/ml proteinase K for 10 min (human sections) in 50 mM Tris-HCl, 5 mM EDTA pH 8.0.

Before use, the probes were denatured by heating to 80° C. for 3 min.

The hybridization buffer consisted of 0.3 M NaCl, 10 mM Tris-HCl, 10 mM NaH2PO4, 5 mM EDTA, 0.02% (w/v) Ficoll 400, 0.02% (w/v) polyvinyl pyrrolidone (PVP)-40, 0.02% (w/v) bovine serum albumin (BSA) fraction V, pH 6.8, 50% formamide, 10% dextran sulphate, 0.92 mg/ml t-RNA, 8.3 mM dithiothreitol (DTT).

In all steps, diethyl pyrocarbonate-treated water (DEPC-H2O) was used. The sections were incubated overnight at 55° C. with sense or anti-sense probes in a moist chamber containing DEPC-H2O. After hybridization, the sections were washed under increasing stringency at 55° C. in 2× sodium chloride-sodium citrate (SSC), 1×SSC, and 0.2×SSC containing 0.1% SDS and 10 mM DTT. The sections were treated with RNase A (20 μg/ml) for 10 min in NTE buffer (0.5 M NaCl, 10 mM Tris-HCl, pH 7.2, 1 mM EDTA), washed in 0.2×SSC, 10 mM DTT, and dehydrated in ethanol with 0.3 M ammonium acetate. The sections were coated in liquid photo emulsion from Ilford (Marly, Switzerland) and stored in the dark at 4° C. After 3 weeks, the sections were developed using D-19 (Sigma, St. Louis, Mo., USA) and counterstained with Mayer's Hematoxylin (Sigma).

### Tissue Arrays and Other Tissue Samples.

Four commercially available bladder cancer tissue arrays were examined. To correlate the expression of ADAM12 with tumor grade, three tissue arrays (BC12011, BL801, and BC12012) were obtained from Biomax, Inc. (Rockville, Md., USA).

A total of 155 cases (age range: 38-88 years old, 46 females and 109 males) were examined: 18 grade 1 tumor cases, 54 grade 2 tumor cases, and 83 grade 3 tumor cases. The histopathological entities included 152 transitional cell carcinomas, 1 squamous carcinoma, and 2 adenocarcinomas. To correlate the expression of ADAM12 with tumor stage, an AccuMax array (A215-urinary bladder cancer tissues) was obtained from ISU (ISU ABXIS Co., Stretton Scientific Ltd. Derbyshire, UK). This array contained 45 cancer cases, with two spots for each cancer case, and four non-neoplastic cases with one spot each. Forty of the cases were classified according to the tumor-node-metastasis (TNM) system, and found to be Ta (eight cases), T1 (14 cases), T2 (seven cases), T3 (six cases), and T4 (five cases).

Histological grading of these 40 cases demonstrated five grade 1 tumor cases, 14 grade 2 tumor cases, and 21 grade 3 tumor cases. The pathological entities included 34 transitional cell carcinomas, four squamous carcinomas, and two adenocarcinomas. Two cases were not classified according to TNM, and three cases were diagnosed as carcinoma in situ. For the 40 classified cases, there were 10 female and 30 male patients (age range: 33-87 years old). Tissue specimens were fixed in formalin, embedded in paraffin, and spots 1 mm in diameter used for tissue arrays. Adjacent nontumorous tissue present in some of the cases on the arrays was also examined, as were tissue specimens of normal bladder mucosa from 10 persons without bladder cancer.

### Antibodies

Antibodies against human ADAM12 used in this study were a rabbit antiserum against the recombinant cysteine-rich domain (rb122), a rabbit antiserum against the recombinant prodomain (rb132), a rabbit antiserum against a carboxy-terminal ADAM12-S peptide (rb116), a rabbit antiserum against a carboxy-terminal ADAM12-L peptide (rb109), a rat monoclonal antibody recognizing the disintegrin domain of ADAM12 (2E7), and mouse monoclonal antibodies recognizing ADAM12 (6E6, 8F8, and 6C10). Antibodies to uroplakin 3 (AU1) were obtained from American Research Products (Belmont, Mass., USA).

### Immunostaining

Tissue sections were deparaffinized, treated with 0.1% hydrogen peroxide for 10 min to inhibit endogeneous peroxidase, treated with 5 μg/ml proteinase K for 10 min in 50 mM Tris-HCl, pH 7.5, and incubated with polyclonal antibodies to human ADAM12 or uroplakin 3 (1:200 in Dulbecco's Phosphate-Buffered Saline with no calcium and magnesium (PBS)) in a moist chamber for 1 hr at room temperature.

Urine samples were mixed with equal amounts of 99% ethanol, centrifuged for 2 min in a Cytospin microfuge (Shandon, Pittsburgh, Pa., USA) to collect cells onto glass slides, and the cells air-dried. Cells were subsequently permeabilized with 0.2% Triton X-100 in PBS for 5 min at room temperature and incubated with rb122 (1:200) or uroplakin 3 (1:150) for 1 hr at room temperature. Detection was performed using the DakoChemMate detection kit (DAKO, Glostrup, Denmark), which is based on an indirect streptavidin-biotin technique using a biotinylated secondary antibody.

As a negative control, primary antibodies were either omitted or replaced with non-immune rabbit or mouse serum as described. All such control sections were negatively stained. Tumor cells were rated ADAM12-positive when the immunostaining reaction was clearly above the negative background. Cells were examined using a Zeiss Axioplan microscope connected to an AxioCam camera using the AxioVision software.

### Western Blotting of Urine Samples

Urine samples from bladder cancer patients whose tumors had been analyzed by microarray were also analyzed by Western blotting. Urine was collected from 11 patients with Ta tumors (one grade 1 tumor case, nine grade 2 tumor cases, and one grade 3 tumor case), four patients with T1 (all being grade 3 cases), and 17 patients with T2-4 tumors (16 grade 3 tumor cases and one grade 4 tumor case).

In addition, urine from six patients with non-muscle invasive bladder tumors was collected at three time points: a) prior to transurethral resection; b) during the surveillance period in which no tumor could be detected; and c) when recurrence of invasive tumor was diagnosed.

Urine samples were collected immediately into sterile containers before surgery or control cytoscopy and centrifuged, and the pellets and supernatants frozen at −80° C. Samples containing blood were excluded.

Cytology specimens were assessed and considered positive only when malignant cells were present. Urine samples from eight volunteers (Caucasians) with no history of bladder tumors (age range: 25-65 years old) served as normal standards. Normal standard specimens were selected to evaluate the specificity of the Western blot and included five cases of benign prostatic hyperplasia and two cases of pregnancy. To reduce the amount of albumin, all urine samples were absorbed with Fast flow cibacron blue 3GA (Sigma) for 3 hr at 4° C. before analyses.

Protein concentration was measured using the BCA protein assay kit (Pierce, Rockford, II, USA). Urine samples (40 μg) or purified ADAM12-S (28,29) were boiled in SDS sample buffer with (reducing) or without DTT (nonreducing) and resolved by NuPAGE 12% Bis-Tris gels (Invitrogen), followed by electrophoretic transfer to Immobilon-P membranes (polyvinylidene difluoride [PVDF] membranes from Millipore Corp. Billerica, Mass., USA). Membranes were blocked overnight with 5% nonfat dried milk at 4° C., then incubated with primary polyclonal or monoclonal antibodies against human ADAM12. Horseradish peroxidase (HRP)-conjugated goat anti-rabbit IgG and goat anti-mouse IgG were used as secondary antibodies. Chemiluminescent detection of HRP was performed by standard methods (Amersham Corp.).

The densities of the observed 68 kDa band were estimated from films using the NIH Image 1.61 program (http://rsb.info.nih.gov/nih-image). Urine from each of the volunteers was pooled and used as a normal standard on each of the Western blots. The densitometric score of the pooled normal standard was used to normalize the apparent amount of the ADAM12 68 kDa band in urine from normal individuals and cancer patients. In some experiments, immunoprecipitation of 500 μl aliquots of urine supernatant was performed as described using a mixture of mouse monoclonal antibodies (6E6, 8F8, and 6C10) (30, 31) and subjected to Western blot as described above.

### Statistical Analysis

Statistical analysis was done using the Mann Whitney test, the Student's t-test or the Chi-square (Pearson). P-values <0.05 were considered statistically significant—but any analysis know to the skilled addressee may be used.

### Example 2

### ADAM8, 10, and 12 Gene Expression in Bladder Cancer Correlates with Disease Status

Gene expression profiling was performed using a customized Affymetrix GeneChip array. This GeneChip contained probe sets for specific detection of 18 different ADAM transcripts (ADAM2, 3a, 5, 8, 9, 10, 11, 12, 15, 19, 20, 22, 23, 28, 29, 30, 32, and 33).

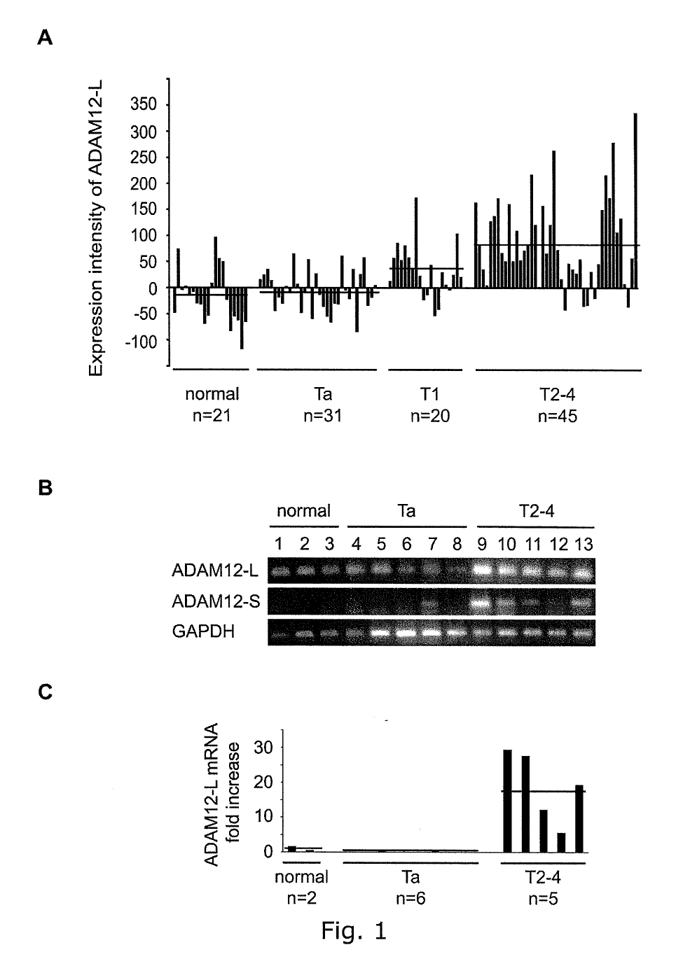

The present inventors found that only ADAM8, 10, and 12 had a positive correlation between gene expression and the disease stage of bladder cancer (FIG. 1A and supplemental FIG. 1). In the present study the present inventors subsequently focused only on the expression of ADAM12 in bladder cancer.

The GeneChip contained transcript variants of both ADAM12-L and ADAM12-S. ADAM12-L was expressed at low levels in normal bladder biopsies and Ta tumors (average expression intensity: −17 and −6, respectively), higher levels in T1 tumors (average expression intensity: 33), and at the highest levels in T2-4 tumors (average expression intensity: 89) (FIG. 1A).

The present inventors found a highly significant difference between the expression of ADAM12-L in normal tissue and Ta tumors compared to T1 tumors (p=0.00074, Student's t-test) and to T2-4 tumors (p=1.0×10-10). ADAM12-S transcripts were not detected in the bladder tumors using this array.

To confirm and quantitate the presence of ADAM12-L and -S mRNA in tumor tissue from a subset of the patients analyzed by microarray (three normal, five Ta, and five T2-4), RT-PCR and qPCR were performed. Using RT-PCR, ADAM12-L was detected in all samples and ADAM12-S was largely present in the T2-4 tumor samples (FIG. 1B). The PCR products were sequenced, and comparison of the sequences to the GenBank verified the identity of nt 2378-2512 in ADAM12-S (AF023477) and nt 2816-2916 in ADAM12-L (AF023476). The present inventors developed a method for qPCR for ADAM12-L and used the method to analyze a subset of the patients analyzed by microarray (two normal, six Ta, and five T2-4). ADAM12-L mRNA was expressed at approximately 15-fold higher levels in T2-4 tumors compared to normal tissue (FIG. 1C; p=0.017, Student's t-test).

### Example 3

### ADAM12 Gene Expression in Bladder Cancer is Concentrated in Tumor Cells

Single-stranded sense and anti-sense ³⁵S labeled RNA probes were generated by in vitro transcription of ADAM12 cDNA and used for in situ hybridization on tumors obtained from the MMTV-PyMT mouse breast cancer model in which transgenic human ADAM12 is expressed.

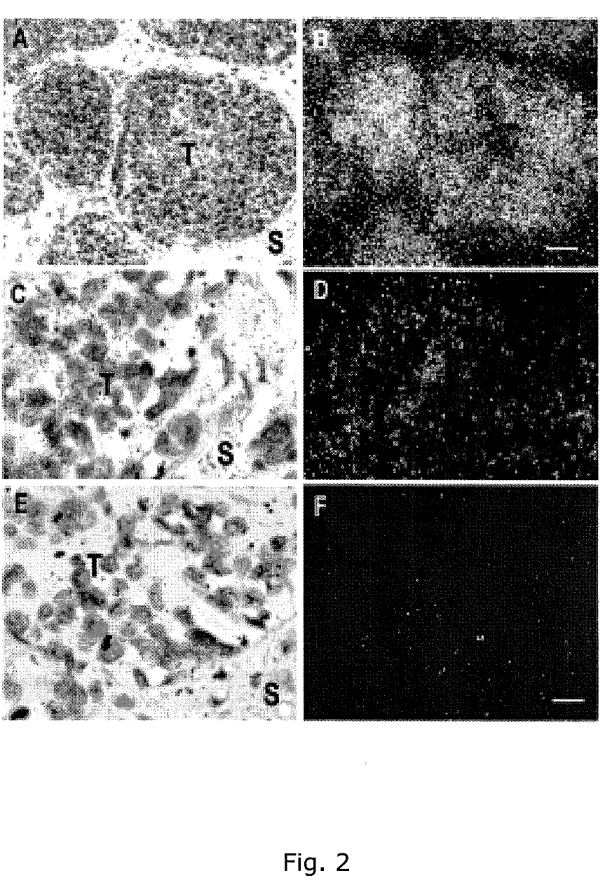

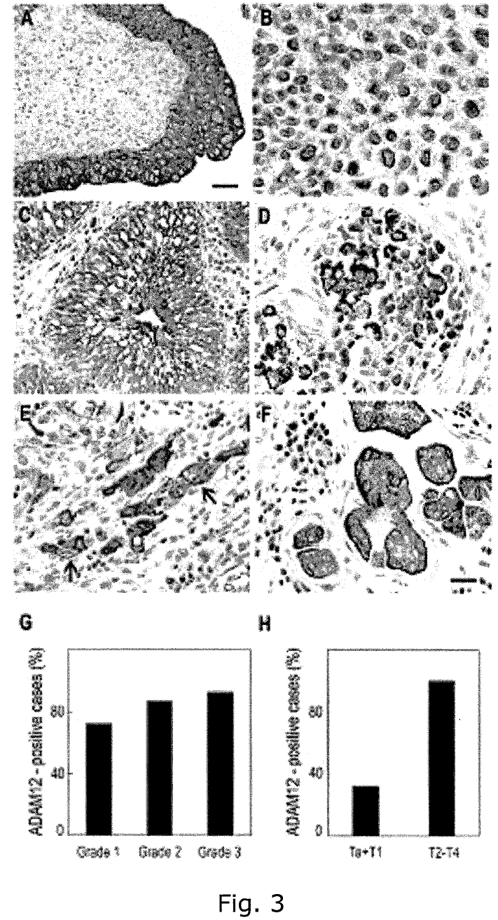

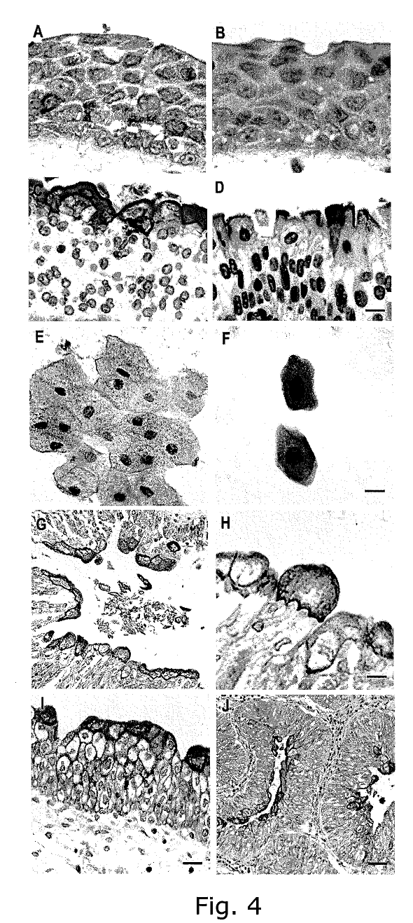

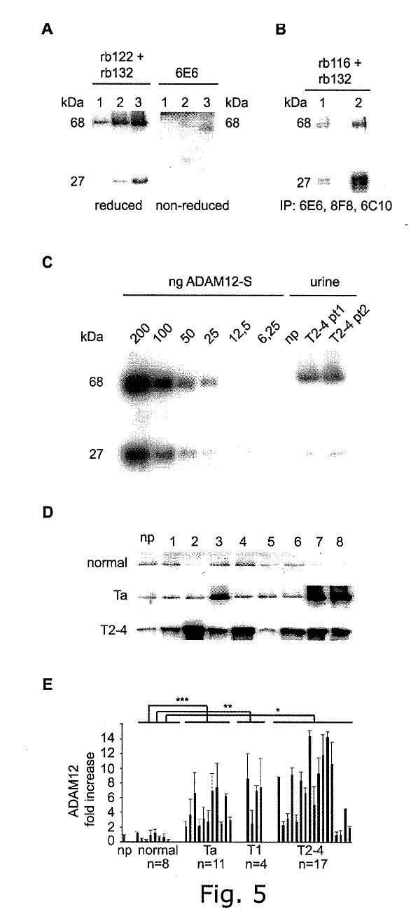

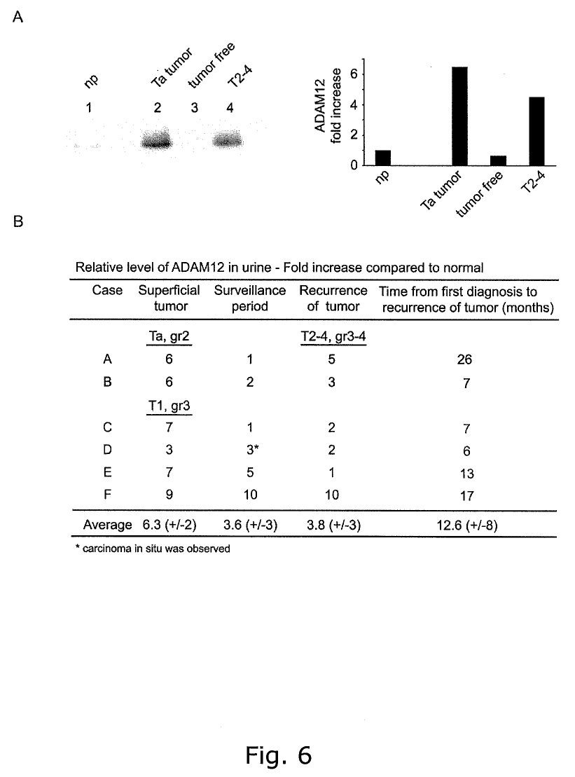

Intense positive signals for ADAM12 were found in the murine breast carcinoma cells with the anti-sense probes (FIG. 2A,B).

The sense probes gave only a background signal.

This result confirmed the specificity of the probes for human ADAM12. These probes were subsequently used to examine ADAM12 mRNA expression in human bladder cancer tissue (FIG. 2C-F). Positive signals for ADAM12 were found in the tumor cells in all grades with the anti-sense probes, while lower signals were observed in the surrounding stroma (FIG. 2C,D).

Much lower levels of signals were found with the sense probes in either the tumor cells or in the surrounding stroma (FIG. 2E,F). These results confirm that ADAM12 mRNA is expressed in human bladder cancer and is located primarily in the tumor cells.

### Example 4

### ADAM12 Immunostaining Correlates with Tumor Grade and Stage

The distribution of ADAM12 protein in bladder cancer tissue was evaluated by immunohistochemistry on tissue arrays (FIG. 3A-F).

In most cases, tumor cells exhibited strong immunostaining. Areas representing apparent invasive fronts appeared to be most intensely stained (FIG. 3E) and strongly positively stained tumor cells could be seen in small blood vessels (FIG. 3F). A few occasional stromal cells exhibited immunostaining.

To evaluate the correlation between ADAM12 protein expression and tumor grade (histological criteria), 155 cases of bladder carcinomas from three different tissue arrays were immunostained. Samples from a great majority of the cases (87%, 135 cases) exhibited positive ADAM12 immunostaining. More specifically, 93% (77 cases) of grade 3, 85% (46 cases) of grade 2, and 72% (12 cases) of grade 1 tumor samples were positive for ADAM12 (FIG. 3G). The difference between the number of grade 3 and the number of grade 1 tumors positive for ADAM12 staining was found to be statistically significant (p<5×10-3; Chi-square; Pearson).

To evaluate the correlation between ADAM12 expression and tumor stage, a tissue array with 40 cases staged according to the TNM system was evaluated (FIG. 3H). All the T2-4 tumors (18 cases) exhibited ADAM12 positive staining, while only 32% of the Ta+T1 tumors (22 cases) were immunoreactive for ADAM12 (p<1×10-5; Chi-square; Pearson).

### Example 5

### Distinct ADAM12 Immunostaining of Umbrella Cells in the Normal Mucosa

ADAM12 protein expression was examined in adjacent nontumorous mucosa and mucosa from patients without bladder cancer. In most cases, the normal urothelium stained very weakly (FIG. 4A).

Interestingly, the so-called umbrella cells often exhibited intensely positive ADAM12 staining. ADAM12 was located both intracytoplasmically and along the cell membrane in these cells (FIG. 4C).

The identity of these cells as umbrella cells was confirmed by immunostaining with antibodies to uroplakin 3 (FIG. 4D), an umbrella cell marker. Umbrella cells shed into the urine were also immunoreactive with antibodies to ADAM12, whereas squamous epithelial and other urothelial cells were negative or only weakly positive (FIG. 4E,F).

Interestingly, urothelium with atypic or dysplastic characteristics demonstrated increased positive cytoplasmic ADAM12 immunoreactivity (FIG. 4G-I). Finally, the present inventors found that “umbrella-like” differentiated tumor cell in the bladder cancer tissue exhibited striking ADAM12 immunoreactivity (FIG. 41).

### Example 6

### ADAM12 is Detected in the Urine from Bladder Cancer Patients

Purified human ADAM12-S appears as two separate bands on SDS-PAGE. The 68 kDa band represents the metalloprotease, disintegrin, cysteine-rich, and EGF-like domains and the 27 kDa band represents the prodomain that remains non-covalently associated with the body of the molecule following furin cleavage.

Urine from bladder cancer patients was subjected to Western blotting analysis using a series of different ADAM12 domain-specific antibodies. Polyclonal antibodies to the cysteine-rich domain (rb122) recognized the 68 kDa band, while polyclonal antibodies to the prodomain (rb132) recognized the 27 kDa band (FIG. 5A).

Under nonreducing conditions, a monoclonal antibody against ADAM12 (6E6) detected a protein band migrating slightly faster than the 68 kDa protein as previously reported (35).

In addition, monoclonal antibodies to the disintegrin domain (2F7) reacted with the 68 kDa band and occasionally to a 50 kDa band that appears to be a degradation product.

Immunoprecipitation of urine supernatant using monoclonal antibodies against ADAM12, followed by immunoblotting with polyclonal antibodies specific for the carboxy-terminus of ADAM12-S (rb116) and for the prodomain and (rb132) detected ADAM12-S in the urine of bladder cancer patients (FIG. 5B).

To determine the approximate level of ADAM12 in urine from healthy individuals and cancer patients, the present inventors compared the amount of ADAM12 in urine with a standard of purified ADAM12-S (FIG. 5C).

Using densitometric quantitation of the 68 kDa band, the present inventors found approximately 4-10 μg ADAM12/ml urine in cancer urine.

In normal urine, ADAM12 was only weakly detected i.e. less than 1 μg/ml urine (FIG. 5D,E).

To further quantitate the relative amount of ADAM12 in cancer urine compared to urine from healthy controls, the present inventors examined 32 samples (11 Ta, 4 T1, and 17 T2-4) of cancer urine and eight samples of healthy control urine by Western blotting and densitometric quantitation (FIG. 5E).

Importantly, the relative amount of ADAM12 protein was significantly higher in urine from patients with a Ta tumor (approximately four-fold increase; p 0.0002, Student's t-test), T1 tumor (approximately six-fold increase; p=0.0001, Student's t-test) or with an invasive bladder tumor (T2-T4; approximately seven-fold increase; p=0.0004, Student's t-test) than in urine from normal individuals.

The present inventors also compared the relative level of ADAM12 mRNA from the microarray experiments with the apparent level of ADAM12 protein in the urine, but found no correlation (data not shown).

Routine cytology was performed on 29 bladder cancer cases, and identified 86% of the bladder cancers (Table 1). The level of ADAM12 in the urine of these 29 cases was examined by Western blot. The present inventors chose to use a >2-fold increase in the relative level of ADAM12 compared to normal control by Western blot as “positive.” The relative levels of ADAM12 alone detected 97% (28/29) of the bladder cancers.

In combination with cytology, the relative level of ADAM12 detected 100% of the tumor cases.

Importantly, ADAM12 detected 100% of the Ta and T1 tumor cases, as well as 100% of the grade 2 tumors, while cytology only detected 78% of Ta, 75% of T1, and 78% of grade 2 tumors. To evaluate the specificity of the Western blot, the present inventors analyzed urinary levels of ADAM12 obtained from five cases of benign prostatic hyperplasia and two cases of pregnancy. The level of ADAM12 in the urine of these cases did not differ from the control healthy individuals.

### Example 7

### ADAM12 in the Urine of Bladder Cancer Patients who Underwent Surgical Removal of Tumor Correlates with the Presence of Tumor

The present inventors analyzed two cases of Ta and four cases of T1 tumors that all eventually progressed to the T2-4 stage.

In all tested cases, ADAM12 was detectable in the urine prior to surgery. FIG. 6A illustrates a patient follow-up with decreasing urinary level of ADAM12 after removal of Ta tumor and increasing level with recurrence of invasive tumor. In both Ta tumor cases and in one T1 case, the level of urine ADAM12 decreased following removal of the tumor, and increased again with appearance of invasive tumor (FIG. 6B, case A, B, C). In one case, the urinary level of ADAM12 did not decrease during the period of surveillance; however, selected site biopsies from this patient showed carcinoma in situ (FIG. 6B, case D).

### REFERENCES

WO 06/121710
Mochizuki S, Okada Y. ADAMs in cancer cell proliferation and progression. Cancer Sci. 2007 May; 98(5):621-8. Epub 2007 Mar. 9. PMID: 17355265 [PubMed—in process]
Kveiborg M, Frohlich C, Albrechtsen R, Tischler V, Dietrich N, Holck P, Kronqvist P, Rank F, Mercurio A M, Wewer U M. A role for ADAM12 in breast tumor progression and stromal cell apoptosis. Cancer Res. 2005 Jun. 1; 65(11):4754-61. PMID: 15930294 [PubMed—indexed for MEDLINE]
Carl-McGrath S, Lendeckel U, Ebert M, Roessner A, Rocken C. The disintegrin-metalloproteinases ADAM9, ADAM12, and ADAM15 are upregulated in gastric cancer. Int J Oncol. 2005 January; 26(1):17-24. PMID: 15586220 [PubMed—indexed for MEDLINE]
Roy R, Wewer U M, Zurakowski D, Pories S E, Moses M A. ADAM 12 cleaves extracellular matrix proteins and correlates with cancer status and stage. J Biol Chem 2004; 279:51323-30.
Dyrskjot L, Thykjaer T, Kruhoffer M et al. Identifying distinct classes of bladder carcinoma using microarrays. Nat Genet 2003; 33:90-6.
Dyrskjot L, Zieger K, Kruhoffer M et al. A molecular signature in superficial bladder carcinoma predicts clinical outcome. Clin Cancer Res 2005; 11:4029-36
Junker N, Johansen J S, Andersen C B, Kristjansen P E. Expression of YKL-40 by peritumoral macrophages in human small cell lung cancer. Lung Cancer 2005; 48:223-31.

What is claimed is:

1. A method for screening an individual for bladder cancer said method comprising the steps of:
a) obtaining a sample from the individual,
b) determining the level of ADAM12 in said sample,
c) comparing said level with a reference level,
d) identifying whether the level is different from said reference level; and
evaluating the disease status of the individual or whether the individual has an increased risk of bladder cancer, if the level is higher than the reference level.

2. A method for determining whether an individual is likely to have recurrent bladder cancer after being treated for bladder cancer, said method comprises the steps of:
a) determining the level of ADAM12 in a sample obtained post-treatment from the individual treated for bladder cancer
b) comparing said level with a reference level;
c) identifying whether the level is different from said reference level; and
evaluating whether the individual is likely to have recurrent bladder cancer, if the level is higher than the reference level.

3. The method according to claim 1, wherein the level of ADAM12 is a combined level of ADAM12 selected from the group consisting of any combination of the level of ADAM12 polypeptide, the level of polynucleotide encoding ADAM12 and the level of specific ADAM12 protease activity.

4. The method according to claim 1, wherein said method can differentiate between different grades and stages of bladder cancer.

5. The method according to claim 1, wherein the ADAM12 level is determined by determining ADAM12-L.

6. The method according to claim 1, wherein the ADAM12 level is determined by determining ADAM12-S.

7. The method according to claim 1, wherein the ADAM12 level is determined by measuring ADAM12 mRNA or DNA.

8. The method according to claim 1, wherein the ADAM12 level is determined on protein level.

9. The method according to claim 1, wherein the sample is urine, blood or plasma.

10. The method according to claim 1, wherein the sample is a tissue biopsy.

11. The method according to claim 1, wherein said determination of the level is carried out on a DNA array.

12. The method according to claim 1, wherein the ADAM12 level is combined with values from at least one other combinatorial marker.

13. The method according to claim 1, wherein the combinatorial marker is selected from the group consisting of ADAM8, ADAM10, MMP2 and MMP9.

14. An array comprising a nucleic acid, which binds ADAM12 for the determination of bladder cancer.

15. A method for treating bladder cancer comprising:
identifying a mammal expressing elevated levels of ADAM12, and
administering to said mammal an effective amount of a drug sufficient to reduce tumor growth or prevent metastasis.

16. A method for screening an individual for the presence of an epithelial cancer said method comprising the steps of:
a) obtaining a sample from the individual,
b) determining the level of ADAM12 in said sample,
c) comparing said level with a reference level,
d) identifying whether the level is different from said reference level; and
evaluating the disease status of the individual or whether the individual has an increased risk of an epithelial cancer, if the level is higher than the reference level.


SEQUENCE LISTING (10 sequences)

SEQ ID NO: 1 - DNA, 19 bp, Artificial Sequence
  Synthetic DNA primer targeting ADAM12-L, forward
cagccaagcctgcacttag

SEQ ID NO: 2 - DNA, 20 bp, Artificial Sequence
  Synthetic DNA primer targeting ADAM12-L, reverse
agtgagccgagttgttctgg

SEQ ID NO: 3 - DNA, 20 bp, Artificial Sequence
  Synthetic DNA primer targeting ADAM12-S, forward
gctttggaggaagcacagac

SEQ ID NO: 4 - DNA, 20 bp, Aritficial Sequence
  Synthetic DNA primer targeting ADAM12-S, reverse
tcagtgaggcagtagacgca

SEQ ID NO: 5 - DNA, 22 bp, Artificial Sequence
  Synthetic DNA primer targeting GAPDH, forward
aaggtcatcccagagctgaacg

SEQ ID NO: 6 - DNA, 20 bp, Artificial Sequence
  Synthetic DNA primer targeting GAPDH, reverse
tgtcataccaggaaatgagc

SEQ ID NO: 7 - DNA, 20 bp, Artificial Sequence
  Synthetic DNA primer targeting 18S rRNA, forward
cgccgctagaggtgaaattc

SEQ ID NO: 8 - DNA, 19 bp, Artificial Sequence
  Synthetic DNA primer targeting 18S rRNA, reverse
ttggcaaatgctttcgctc

SEQ ID NO: 9 - DNA, 50 bp, Artificial Sequence
  Synthetic DNA primer containing T7 recognition site, targeting ADAM12-L, forward
ggatccaataatacgactcactatagggagaggcacaaagtgtgcagatg

SEQ ID NO: 10 - DNA, 50 bp, Artificial Sequence
  Synthetic DNA primer containing T3 recognition site, targeting ADAM12-L, reverse
gagaattcattaaccctcactaaagggagagtctgtgcttcctccaaagc